(19) United States
(12) Patent Application Publication
(10) Pub. No.: US 2002/0072712 A1
(43) Pub. Date: Jun. 13, 2002

(54) Medical wire introducer and protective sheath

(76) Inventors: Jeffrey A. Nool, San Jose, CA (US); Scott A. McGill, Redwood City, CA (US); Mukund R. Patel, San Jose, CA (US); Arlene L. Errazo, Suunyvale, CA (US); George Tsai, Sunnyvale, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/976,847

(22) Filed: Oct. 12, 2001

Related U.S. Application Data
(60) Provisional application No. 60/240,591, filed on Oct. 12, 2000.

(51) Int. Cl. A61M 5/178

(52) U.S. Cl. 604/167.01; 604/164.08

(57) ABSTRACT

A protective sheath assembly is provided that combines the functions of an introducer, a protective sheath and a sealing valve in an integrated assembly. The protective sheath assembly generally comprises a protective sheath with a connecting member or a hub. The protective sheath has a proximal end, a distal end, and an elongated hollow body defining a lumen along the longitudinal axis. The protective sheath accommodates and protects a medical device during insertion through a hemostasis valve into a patient's vasculature. The protective sheath also provides a seal around the medical device to prevent blood back flow.

### RELATED APPLICATIONS

The present application claims priority under 35 U.S.C. 119(e) to U.S. Provisional Application No. 60/240,591, filed Oct. 12, 2000.

### BACKGROUND OF THE INVENTION

1. Field of Invention

The present invention relates to the field of medical wire introducers. More particularly, the invention relates to a medical wire introducer and protective sheath assembly adapted to facilitate the introduction of an intravascular device into a blood vessel. The invention also relates to methods of constructing and using the same.

2. Description of Related Art

Minimally invasive surgical techniques, including intravascular techniques such as angioplasty, have had a rapid development and have gained wide acceptance within the medical fields. In these surgical procedures, a percutaneous or arterial sheath is typically introduced through a puncture or an incision in the patient's skin to provide percutaneous access to blood vessels. A catheter is then inserted through the arterial sheath and is advanced through the arteries to the target site. The catheter can be equipped with a wide variety of devices at its distal end, such as, for example, a balloon. These devices can serve numerous functions, such as occluding the vessel so as to block blood flow, and dilating occluded blood vessels during angioplasty. Solid or hollow thin wires, called guidewires, are commonly used to facilitate the advancement of the catheter through the patient's vasculature to the target site.

Angioplasty balloon catheters can be roughly divided into three categories: over-the-wire (OTW) systems, single-operator-exchange (SOE) or monorail systems, and fixed-wire systems (also called “balloon-on-a-wire”). In an OTW system, a solid guidewire is used to guide a balloon catheter, which is tracked coaxially over the guidewire and can be moved relative to it. SOE balloon catheters are modified OTW catheters, i.e., only the distal portion of a SOE balloon catheter tracks coaxially over the guidewire. In a fixed-wire system, a hollow guidewire is in fluid communication with a balloon mounted at its distal end to supply inflation fluid to the balloon. The guidewire typically has a soft tip at its distal end to prevent damaging tissue during advancement of the catheter through a blood vessel.

Vascular access may include a femoral approach, a brachial approach or a radial approach. A commonly adopted procedure for intravascular surgery through a femoral approach typically involves the following steps: (a) identifying the femoral artery and administrating local anesthetic to the patient; (b) inserting a needle into the femoral artery (or an appropriate peripheral blood vessel) and waiting for blood to flow out through the needle; (c) introducing a guidewire into the blood vessel through the needle and then removing the needle leaving the guidewire in place within the blood vessel; (d) tracking an arterial (percutaneous) sheath and dilator over the guidewire into the blood vessel, so that the distal end of the arterial sheath enters the vessel; (e) removing the dilator and the guidewire leaving the arterial sheath in place; (f) introducing a guide catheter over a guidewire through the percutaneous sheath and advancing it around the aortic arch; (g) removing the guidewire; (h) connecting the guide catheter to an inflation device and a steering tool through a Y-adaptor and/or to a manifold assembly through another Y-adaptor, with the manifold assembly usually being connected to a pressure transducer and a syringe; (i) introducing a catheter over a guidewire through the guide catheter and the Y-adaptor, and advancing it through the arteries until the distal end of the catheter reaches the treatment site. Alternatively, if a “balloon-on-wire” catheter, such as a distal occlusion wire (DOW), is being used in the last step, the catheter may be directly inserted into the blood vessel through the guide catheter and Y-adaptor.

While performing intravascular treatments, such as angioplasty, there is the possibility that the treatment may dislodge plaque from the vessel walls. When emboli or other particulates flow downstream to occlude blood flow in smaller vessels, they can cause serious damage, such as stroke. To address this problem, the intravascular surgical procedure described above may include the step of inserting an intravascular occlusion device, such as a distal occlusion wire (DOW) or a filter device through the arterial sheath before the intravascular treatment is performed. Intravascular occlusion devices typically deploy a balloon or a filter provided at the distal end of a catheter downstream of the target site to trap and contain the emboli that may dislodge.

During the process of inserting a catheter through the arterial sheath, blood may bleed back under arterial pressure. In order to avoid excess bleeding and possible air embolisms, a hemostasis valve or a Touhy-Borst valve is usually installed in the Y-adaptor. Various hemostasis valves have been developed and are known in the art. A hemostasis valve is typically composed of one or more resilient, cylindrical pieces formed with slits or holes. These slits and/or holes are sized and configured so that they are normally closed, but may permit an intravascular device to be forced through while maintaining a seal around the device.

However, when a fixed-wire catheter, such as a DOW, is used, it becomes very difficult to insert the catheter through a hemostasis valve because the distal end of a “balloon-on-wire” catheter usually has a soft tip for preventing damage to the blood vessel. A concomitant problem of having a balloon with a soft tip, however, is that the soft tip is capable of being damaged while inserting it through the hemostasis valve. Thus, there is a further need for a protective sheath to shield the balloon and the soft tip when introducing the same through a hemostasis valve.

Presently, either a guidewire needle introducer or a protective sheath, are separately used to introduce a balloon catheter into a guide catheter via a hemostasis valve mounted within the Y-adaptor. However, when using either procedure, the problem of back blood flow still exists as the hollow protective sheath, or the large needle, is inserted into the Y-adaptor. For some catheter devices of the balloon-on-wire type, such as a DOW, these methods, i.e. using either a protective sheath or a large needle introducer, also will be very cumbersome, time consuming, and cause greater back bleed.

Similarly, when a filter device is used, it becomes very difficult to insert the filter device through a hemostasis valve because the filter device may include relatively fragile structure along the distal end that can easily be damaged as it is passed through the valve. There is also a significant problem with blood back flow during the insertion of the filter device because of the difficulty in maintaining a tight seal around the filter and the catheter or shaft carrying the filter.

Thus, there exists an urgent need for an introducer device which can be conveniently and safely utilized to protect a balloon catheter, filter device or any-fixed wire device as it is inserted through a hemostasis valve or a Touhy-Borst. It is desirable that such an introducer device also be capable of maintaining a seal to prevent blood back flow through the valve.

### SUMMARY OF THE INVENTION

A protective sheath assembly is provided for introducing a fixed-wire balloon catheter or a filter device into a blood vessel through a hemostasis valve or a Touhy-Borst valve. Also provided are a wire introducer/protective sheath assembly and methods of constructing and using the same.

A preferred embodiment of the protective sheath assembly combines the function of an introducer with both the function of a protective sheath and the function of a sealing valve, thus forming an integrated protective sheath assembly. The protective sheath assembly comprises a protective sheath having a proximal end, a distal end, and an elongated hollow body defining a lumen along the longitudinal axis. The lumen of the protective sheath further comprises three portions: a proximal portion, a distal portion, and a transitional portion between the two.

Protective Sheath Assembly For Use With a Balloon Catheter

In one aspect, the protective sheath assembly is designed for use with a balloon catheter. The protective sheath assembly has a distal portion with an inner diameter that is larger than the diameter of the balloon, and a proximal portion with an inner diameter that is larger than the outer diameter of the guidewire. The lumen of the protective sheath serves several functions including: protecting the balloon and the soft tip, accommodating movement of the guidewire, and forming a seal around it. It will be appreciated that this protective sheath assembly can also be used with a filter device, as described below.

In one embodiment, the dimension of the lumen of the protective sheath is designed so that the inner diameter of the lumen at its proximal portion is slightly larger than the outer diameter of the guidewire, thus providing adequate and smooth passage of the guidewire. The clearance between the two diameters, or between the inner surface of the proximal portion of the protective sheath and the outer surface of the guidewire, should be small enough so as to substantially minimize back blood flow under arterial pressure. In addition, other sealing mechanisms can be used in the proximal portion. For example, an O-ring or a sealing sleeve can be installed within the proximal portion of the protective sheath through a connecting member or by alternative means. In this case, the protective sheath body and the lumen may have uniform inner and outer diameters. It is also possible to use a reducing member to connect the proximal portion with a smaller dimension to the distal portion with a larger dimension.

At the distal portion of the protective sheath, a larger dimension of the lumen is preferably provided to accommodate and protect the balloon and the flexible tip mounted on distal end of a guidewire. The protective sheath preferably has a flared inner surface and a tapered outer surface at its distal end, thereby permitting easier back load of the guidewire and insertion of the protective sheath through a hemostasis valve to a blood vessel. Overall, a smooth transition of the lumen dimension between the proximal portion is desirable. The smooth loading of the guidewire is facilitated by the relative dimensions of the distal and proximal portions, with the proximal portion usually having a smaller diameter and the distal portion usually having a larger diameter. The longitudinal center line of the lumen of the protective sheath usually is configured as a straight line, but may also be made curved at a desired angle.

In another embodiment, an elongated tubular sleeve is used to enclose the proximal portion of the protective sheath in order to further support and hold the sheath. In this case, the sleeve has an inner diameter slightly larger than the outer diameter of the proximal portion of the protective sheath thereby substantially covering the entire proximal portion at the point where the protective sheath has a smaller outer diameter. The proximal end of the protective sheath and the tubular sleeve is connected to a hub or a connecting member.

In another embodiment, a metal hypotube or a needle is partially inserted into the lumen of the protective sheath from the proximal end to facilitate the smooth movement of the guidewire. The metal hypotube has an inner diameter slightly larger than the outer diameter of the guidewire so that the guidewire can be freely moved through the metal hypotube. The outer diameter of the metal hypotube is slightly larger than the inner diameter of the lumen at the proximal portion of the protective sheath.

The cross-sectional area of the protective sheath is preferably circular, but other shapes, such as an oval shape, are possible. More than one channel can be provided in the protective sheath. The cross-sectional plane at the distal end of the protective sheath assembly also does not have to be perpendicular to the longitudinal axis, and may be of a different shape.

A connecting member or a hub is provided at the proximal end of the protective sheath. The connecting member has a distal side with a first mechanism for receiving the proximal end of the protective sheath and a proximal side with a second mechanism for optionally receiving an introducer.

The protective sheath can be used alone or in combination with an introducer, such as a needle introducer. The introducer has a first end proximal to the operator, a second end for connecting to the protective sheath, and an elongated body defining a cavity along its longitudinal axis for receiving a guidewire. The connection between the introducer and the protective sheath can be made in different ways. In one embodiment, the connecting member is a female luer lock having a proximal end with a cup-shaped cavity for receiving the second end of the introducer, a distal end with an elongated cavity for receiving the proximal end of the protective sheath, and a through-hole aligned with the lumen of the protective sheath and the longitudinal cavity of the introducer. The proximal end of the protective sheath is inserted into the distal end of the female luer lock through the elongated cavity. The second end of the introducer is inserted into the proximal end of the female luer lock through the cup-shaped cavity. It is desirable to make those connections removable by slip-fitting. If necessary, a glue can be used. The through-hole of the female luer lock may taper in diameter (preferably, but not necessarily, in a gradual, uniform manner) from its proximal end to its distal end to facilitate insertion of a catheter. It is also apparent that other locking structures can be used, for example, a ridge-grooved connection.

Protective Sheath Assembly Particularly For Use With a Filter Device

In another aspect, the protective sheath assembly is designed for use with an intravascular occlusion device such as a filter device or other occlusion device. A filter device generally comprises a radially expanding filter located at the distal end of an elongated catheter. The elongated catheter is typically referred to as the filter shaft. The expandable filter is preferably comprised of a flexible material having pores sized to prevent emboli from flowing past the distal end when the filter is expanded in the vasculature of the patient.

The protective sheath assembly designed for use with a filter device generally includes a shaft lumen and a distal lumen. The shaft lumen is located in the proximal portion of the protective sheath and has an inner diameter that is slightly larger than the outer diameter of the filter shaft. The shaft lumen is designed to facilitate smooth movement of the filter while providing a seal against blood back flow. The distal lumen has a larger inner diameter and is designed to accommodate and protect the filter. The protective sheath assembly serves several functions including: protecting the expandable filter as it is passed through a hemostasis valve, accommodating smooth movement of the filter shaft, and forming a seal around the filter shaft to prevent bleed back.

A first embodiment designed for use with a filter device includes an elongated tubular outer sheath that is similar to the protective sheath assembly described above for use with a balloon catheter; however, this embodiment also includes an inner sheath. The inner sheath is inserted into the lumen of the outer sheath and includes the shaft lumen for accommodating the filter shaft. If necessary, the inner sheath may also be formed with a second lumen containing a mandrel to increase the bending stiffness of the inner sheath. The shaft lumen of the inner sheath is formed with smooth walls to facilitate smooth movement of the filter shaft through the shaft lumen.

When the inner sheath is fully inserted into the lumen of the outer sheath, the inner sheath extends from the proximal end of the outer sheath approximately halfway toward the distal end of the outer sheath. The filter shaft may be side loaded into the shaft lumen of the inner sheath by snapping the filter shaft through a slot formed on the side of the inner sheath. The outer diameter of the inner sheath is approximately the same size as the inner diameter of the outer sheath. Therefore, when the inner sheath is inserted into the outer sheath, the inner sheath provides a seal to minimize blood back flow through the lumen of the outer sheath.

The inner sheath preferably includes a proximal hub connected to the proximal end for gripping and retrieving the inner sheath. The proximal hub is formed with a lumen and a slot containing two opposing compressible pads. The compressible pads allow the filter shaft to be pushed through the pads into the lumen when sufficient force is applied. Once the filter shaft is in the lumen, the compressible pads prevent the filter shaft from inadvertently falling out of the lumen. When the inner sheath is fully inserted into the outer sheath, the proximal hub of the inner sheath mates with a hub connected to the proximal end of the outer sheath.

A second embodiment designed for use with a filter device is similar to the embodiment described above, however, in this embodiment, the outer sheath is formed with a proximal slot, a distal slot, and an entrance port between the two slots. The proximal slot and distal slot each extend longitudinally along the outer sheath and allow for side loading and removal of the filter shaft. The entrance port allows the expandable filter at the distal end of the filter device to be inserted through the side of the outer sheath and into the distal lumen of the outer sheath.

The proximal slot in the outer sheath extends longitudinally from the proximal end to the entrance port and is wide enough so the filter shaft may be pushed through the slot. The proximal slot in the outer sheath may be aligned with the slot in the inner sheath so the filter shaft can be side loaded into the shaft lumen of the inner sheath in one single step. After the filter shaft has been loaded into the shaft lumen of the inner sheath, the inner sheath can be rotated relative to the outer sheath. The rotation causes the slots to move out of alignment, thereby locking the filter shaft into the protective sheath assembly.

The distal slot in the outer sheath extends longitudinally from the entrance port to the distal end of the protective sheath. The width of the distal slot is narrower than the width of the proximal slot. The distal slot is provided so the protective sheath may be removed from the filter device by peeling the filter shaft through the proximal and distal slots in the outer sheath.

A third embodiment designed for use with a filter device comprises a protective sheath having a lumen, an entrance port and a single longitudinal slot extending from the entrance port to the distal end. The inner diameter of the lumen is designed to accommodate and protect the expandable filter. This embodiment has no inner sheath, however, may be used in combination with a wide variety of wire introducers that are currently available in the prior art. The wire introducer provides for smooth advancement of the filter shaft through the hemostasis valve.

In use, the filter shaft is first back loaded into the lumen of the wire introducer. The expandable filter is then inserted through the entrance port on the side of the filter sheath and is advanced into the distal portion of the lumen. The distal portion of the filter sheath is then inserted through the hemostasis valve and the expandable filter is advanced through the hemostasis valve into the vasculature of the patient. The filter sheath is then backed out of the valve and the filter sheath is removed by peeling the filter shaft through the longitudinal slot. The wire introducer is then inserted into the hemostasis valve to provide for smooth movement of the filter shaft and to minimize blood back flow.

A fourth embodiment designed for use with a filter device provides a single-piece protective sheath assembly. This embodiment of the protective sheath assembly includes a proximal portion, a distal portion and an entrance port between the proximal and distal portions. The proximal portion is formed with a shaft lumen for accommodating the filter shaft. If necessary, the proximal portion may also be formed with a mandrel lumen to provide structural support. A proximal slot is formed along the side of the proximal portion for providing access to the shaft lumen. The distal portion of the protective sheath assembly is formed with a larger lumen designed to accommodate and protect the expandable filter. The distal portion is formed with a distal slot that allows the filter shaft to be peeled from the protective sheath after the expandable filter has been advanced through the hemostasis valve.

The single-piece protective sheath assembly includes a proximal hub connected to the proximal end for gripping and retrieving the device. The proximal hub is formed with a slot that is aligned with the proximal slot in the protective sheath for inserting the filter shaft into the shaft lumen. The hub slot may contain compressible pads for preventing the filter shaft from falling out of the hub slot.

In another embodiment, the proximal hub may be rotatable relative to the protective sheath for locking the filter shaft into the shaft lumen. After the filter shaft has been inserted into the shaft lumen, the proximal hub may be rotated relative to the protective sheath such that the hub slot and the proximal slot in the protective sheath are moved out of alignment. This effectively locks the filter shaft into the shaft lumen of the protective sheath.

Another embodiment of the proximal hub comprises two separate pieces attached by a hinge. When the hub is open, the lumen in the proximal hub is exposed and the filter shaft can be placed into the lumen. When the hub is closed, the filter shaft is locked into the lumen and cannot be removed.

Another embodiment of the proximal hub comprises two separable halves that can be separated by pulling the two pieces apart to expose the lumen at the center of the protective sheath. When the two pieces of the hub are separated, the filter shaft may be inserted into the lumen at the center of the hub. When the two pieces are pushed back together again, the filter shaft is locked into the lumen of the protective sheath.

### BRIEF DESCRIPTION OF THE DRAWINGS

FIG. 1 is a side cross-sectional view of a protective sheath assembly.

FIG. 2 is a perpendicular cross-sectional view of the protective sheath assembly of FIG. 1.

FIG. 3 is a perpendicular cross-sectional view of the protective sheath assembly of FIG. 1 as seen through sectional line 3-3.

FIG. 4 is an enlargement of the transition section of the protective sheath assembly of FIG. 1 as indicated by line 4-4.

FIG. 5 is an enlargement of the distal end of the protective sheath assembly of FIG. 1 as indicated by line 5-5.

FIG. 6 is a side, partial cross-sectional view of another embodiment of a protective sheath assembly.

FIG. 7 is a perpendicular cross-sectional view of the protective sheath assembly of FIG. 6 as seen through sectional line 7-7.

FIG. 8 is a perpendicular cross-sectional view of the protective sheath assembly of FIG. 6 as seen through sectional line 8-8.

FIG. 9 is a perpendicular cross-sectional view of the protective sheath assembly of FIG. 6 as seen through sectional line 9-9.

FIG. 10A is a side cross-sectional view of the protective sheath assembly according to another embodiment having multiple internal lumen diameters.

FIG. 10B is an enlarged cross-sectional view of the transition region of the protective sheath of FIG. 1A.

FIG. 11 is a schematic representation of a surgery system using a protective sheath assembly to introduce a catheter with balloon into a blood vessel.

FIG. 12 is a side, partial cross-sectional view of a protective sheath assembly according to another embodiment.

FIG. 13 is a top view of the protective sheath assembly according to one embodiment that is well-suited for use with a filter device.

FIG. 14 is a perpendicular cross-sectional view of the distal portion of the outer sheath of FIG. 13 as seen through sectional line 14-14.

FIG. 15 is a perpendicular cross-sectional view of the proximal portion of the outer sheath of FIG. 13 as seen through sectional line 15-15.

FIG. 16 is a perpendicular cross-sectional view of the distal portion of the inner sheath of FIG. 13 as seen through sectional line 16-16.

FIG. 17 is a perpendicular cross-sectional view of the proximal portion of the inner sheath of FIG. 13 as seen through sectional line 17-17.

FIG. 18 is a perspective view of the embodiment shown in FIG. 13 whereby the inner sheath is partially inserted into the lumen of the outer sheath.

FIG. 19 is a top view of the protective sheath assembly according to another embodiment wherein a filter device may be side-loaded into the protective sheath assembly.

FIG. 20 is a perpendicular cross-sectional view of the distal portion of the outer sheath of FIG. 19 as seen through sectional line 20-20.

FIG. 21 is a perpendicular cross-sectional view of the proximal portion of the outer sheath of FIG. 19 as seen through sectional line 21-21.

FIG. 22 is a perspective view of the embodiment shown in FIG. 19.

FIG. 23A is a perspective view of the embodiment shown in FIG. 19 with the slots aligned.

FIG. 23B is a perspective view of the embodiment shown in FIG. 19 with the slots out of alignment.

FIG. 24A is a side end view of the embodiment of FIG. 19 with the slots aligned and the filter shaft in the lumen.

FIG. 24B is a side end view of the embodiment of FIG. 19 with the slots out of alignment and the filter shaft locked in the lumen.

FIG. 25 is a perspective view of the embodiment shown in FIG. 19 with the slots aligned and the filter shaft being peeled out of the protective sheath.

FIG. 26 is a top view of another embodiment of the present invention having an entrance port and a slot in the distal portion.

FIG. 27 is an enlarged perpendicular cross-sectional view of the distal portion of the outer sheath shown in FIG. 26 as seen through sectional line 27-27.

FIG. 28 is an enlarged perpendicular cross-sectional view of the proximal portion of the inner sheath shown in FIG. 26 as seen through sectional line 28-28.

FIG. 29 is a top view of the filter device threaded through an introducer sheath whereby the filter is ready to be inserted into the entrance port in the protective sheath assembly of FIG. 26.

FIG. 30 is a top view of the filter device threaded through the introducer sheath and inserted into the protective sheath assembly of FIG. 26 for advancement through a hemostasis valve.

FIG. 31 is a top view of the protective sheath assembly of FIG. 26 removed from the hemostasis valve and the filter shaft being peeled through the slot in the distal portion of the protective sheath assembly.

FIG. 32 is a top view of the introducer sheath inserted into the hemostasis valve with the protective sheath assembly removed.

FIG. 33 is a top view of another embodiment wherein the protective sheath assembly comprises a single sheath having a longitudinal slot and a proximal hub.

FIG. 34 is a perpendicular cross-sectional view of the proximal portion of the protective sheath assembly of FIG. 33 as seen through sectional line 34-34.

FIG. 35 is a perpendicular cross-sectional view of the proximal hub of the protective sheath assembly of FIG. 33 as seen through sectional line 35-35.

FIG. 36 is a perspective view of the embodiment of the protective sheath assembly shown in FIG. 33.

FIG. 37A is a top view of another embodiment of the single-piece protective sheath assembly of FIG. 33 having a rotating proximal hub.

FIG. 37B is a side view of the rotating proximal hub shown in FIG. 37A with the slots out of alignment.

FIG. 38A is a cross-sectional view of the rotating proximal hub of FIG. 37A with the slots in alignment.

FIG. 38B is a cross-sectional view of the rotating proximal hub of FIG. 37B with the slots out of alignment.

FIG. 39 is a perspective view of the embodiment shown in FIG. 37A whereby the slots in the rotating hub are not in alignment.

FIG. 40 is an exploded perspective view of one embodiment of the rotating proximal hub.

FIG. 41 is an exploded perspective view of another embodiment of the rotating proximal hub.

FIGS. 42A and 42B show a perspective view of a hinged proximal hub.

FIGS. 43A and 43B show a perspective view of a separable proximal hub.

FIG. 44A is a perspective view of a rotating proximal hub.

FIG. 44B is an enlarged perspective view of a rotating proximal hub wherein the slot in the hub is aligned with the slot in the sheath.

FIG. 44C is an enlarged perspective view of a rotating proximal hub wherein the slot in the hub is not aligned with the slot in the sheath.

FIG. 44D is an exploded view of the rotating proximal hub shown in FIG. 44A.

FIG. 45 is a perspective view of another embodiment wherein the distal portion of the outer sheath is formed with pores for flushing lumen with fluid to remove air before use.

FIG. 46 is a perspective view of another embodiment wherein the proximal end portion of the protective sheath is formed with a longitudinal slot and lower radial projections for causing the slot to open to access the interior lumen.

FIG. 47 is a cross-sectional view of the protective sheath of FIG. 46 as seen through sectional line 47-47.

FIG. 48 is a perspective view of the protective sheath of FIG. 46 wherein the proximal end of the protective sheath is deformed to open the longitudinal slot for accessing the interior lumen of the protective sheath.

FIG. 49 is an enlarged perspective view of the proximal end of the protective sheath of FIG. 46.

FIG. 50 is a perspective view of a variation of the protective sheath of FIG. 46 wherein the device is formed without upper radial projections.

FIG. 51 is a perspective view of another variation of the protective sheath of FIG. 46 wherein the walls of the protective sheath overlap along the proximal end portion.

FIG. 52 is a cross-sectional view of the protective sheath of FIG. 51 as seen through sectional line 52-52.

FIG. 53 is a perspective view of the protective sheath of FIG. 52 wherein the proximal end of the protective sheath is deformed for accessing the interior lumen of the protective sheath.

FIG. 54 is an enlarged perspective view of the proximal end of the protective sheath of FIG. 53.

FIG. 55 is a perspective view of a variation of the protective sheath of FIG. 51 wherein the device is formed without upper radial projections.

FIG. 56 is a partial sectional view of a shaft and filter subassembly deployed in a blood vessel, as well as a friction fit mechanism located proximal of the filter subassembly.

FIG. 57 is a side view of a strut hypotube of the filter subassembly.

FIG. 58 is a perspective view of the strut hypotube.

FIG. 59 is a sectional view of the strut hypotube, taken along the line 59-59 in FIG. 57.

FIG. 60 is a side view of a pull wire for use in the shaft and filter subassembly.

FIGS. 61 and 62 are partial cross-sectional views of a kink protection system for the pull wire, reflecting system conditions when the filter subassembly is in the contracted and expanded configurations, respectively.

FIGS. 63-65 show an adapter for use with the shaft and filter subassembly of FIG. 56.

FIG. 66 illustrates another embodiment of an adapter for use with the shaft and filter subassembly of FIG. 56.

### DETAILED DESCRIPTION OF THE PREFERRED EMBODIMENTS

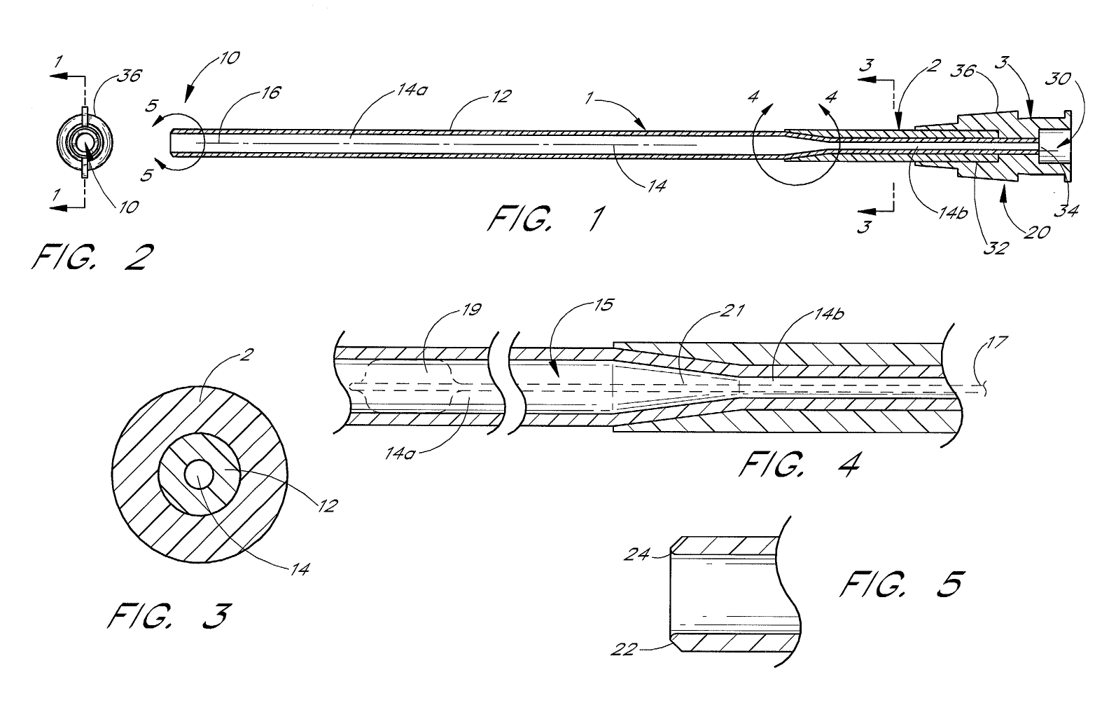

FIG. 1 shows a first preferred embodiment of a protective sheath assembly. As illustrated in FIG. 1, the protective sheath assembly has three major parts: a protective sheath 1, a female luer lock 3, and a strain-relief tubing 2. The protective sheath 1 has an elongated tubular body 12 defining an elongated lumen 14 along a longitudinal axis 16. FIG. 2 is an end view of the sheath 1 of FIG. 1 looking from the distal end 10 toward the proximal end 20, wherein is located the plane 36 to facilitate the handling of the sheath 1. FIG. 3 is a cross-sectional view taken through the proximal end 20 of the sheath 1 and illustrating a strain relief tubing 2 as positioned about the elongated tubular body 12 which defines the lumen 14.

The protective sheath 1, shown in FIG. 1, also has a distal end 10 and a proximal end 20. As shown in FIG. 5, the distal end 10 contains a tapered outer diameter 22 and a flared inner diameter 24. This outer taper 22 configuration facilitates introduction of the sheath 1 into the Touhy adapter, while the inner flare 24 provides easier introduction of the wire 17 into the sheath.

The lumen 14 can be further divided into two portions, the proximal portion 14b with a smaller dimension starting from the proximal end 20 and the distal portion 14a with the longer dimension starting from the distal end 5-5 and extending over a relatively large part of the protective sheath 1. Of course, the proximal end could also be made longer if desired.

The dimension of lumen 14 at the proximal portion 14b may vary depending on the outer diameter of the guidewire to be used. The inner diameter and the length of the proximal portion 14b of lumen 14 is designed so that the guidewire can be moved smoothly through the lumen 14, while providing a good seal between the guidewire and the lumen 14 so as to prevent, or minimize, back blood flow under arterial pressure. The dimension of the distal portion 14a of lumen 14, including the length and the inner diameter, may vary depending on the sizes of the balloon and the soft tip. However, the distal portion of 14a should be large enough to accommodate and protect the balloon, as well as the soft tip of a balloon catheter, or other fixed wire devices.

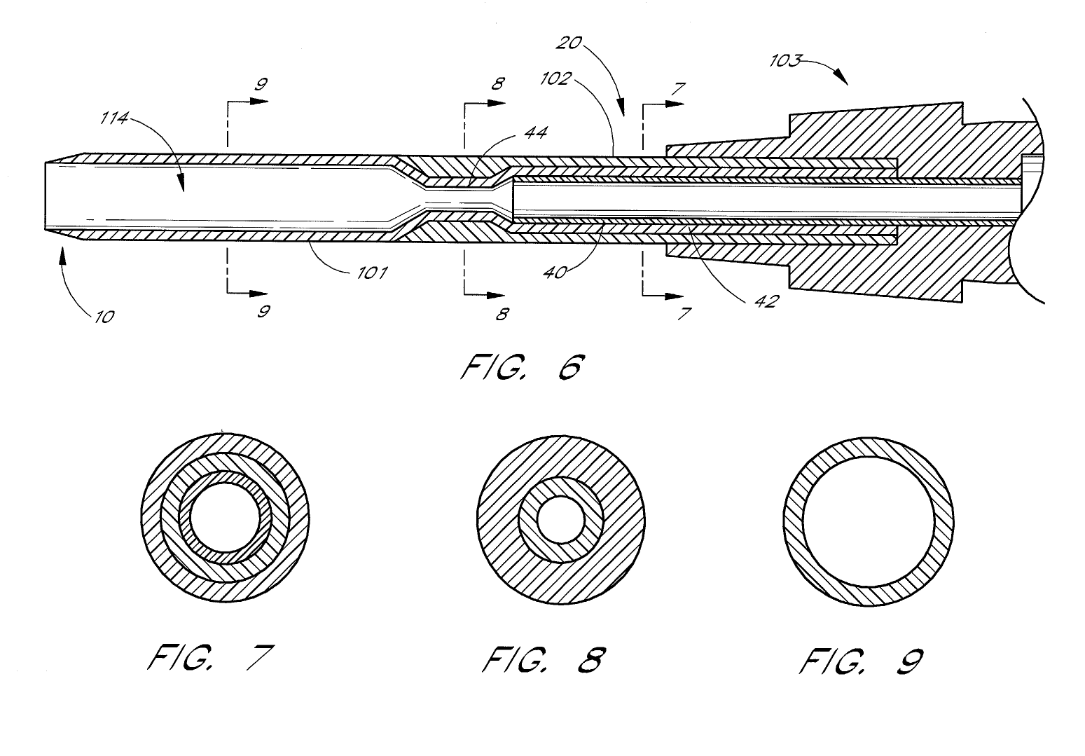

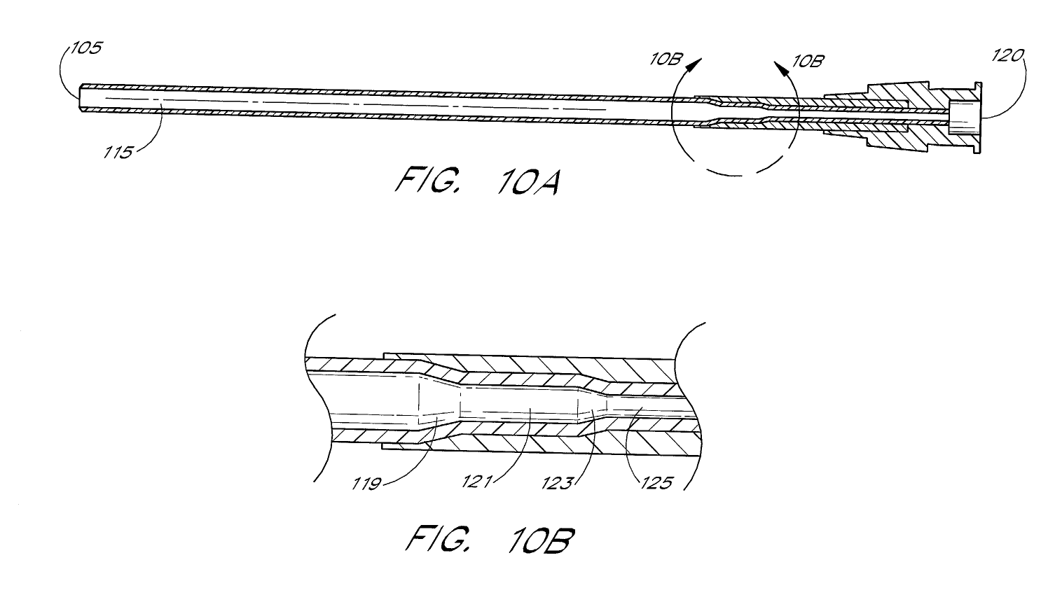

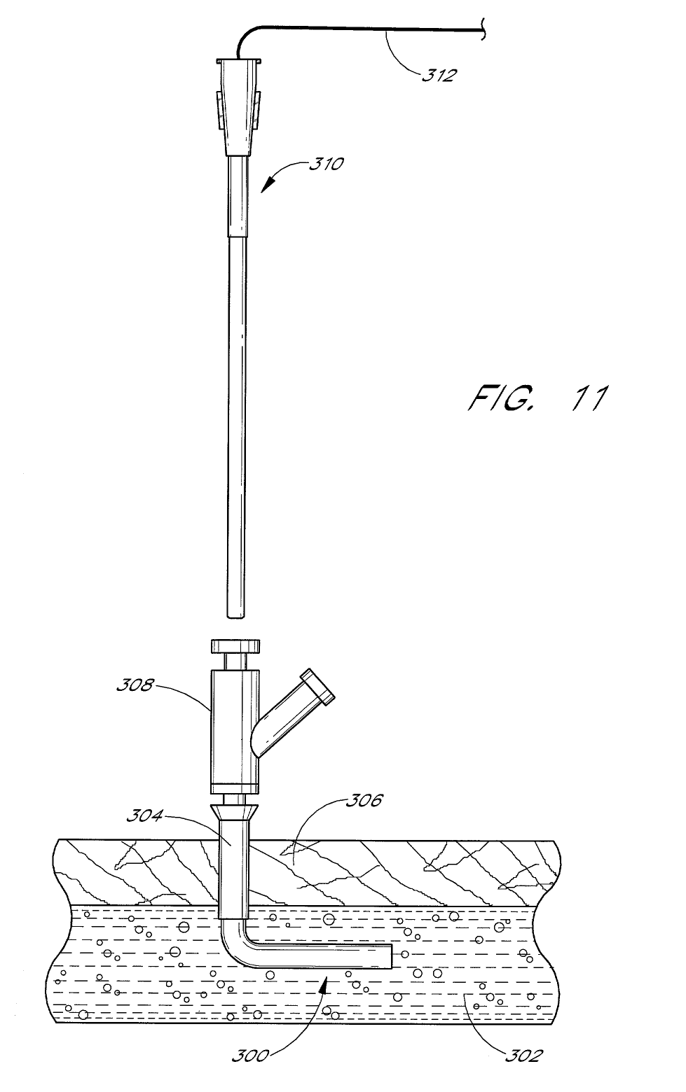

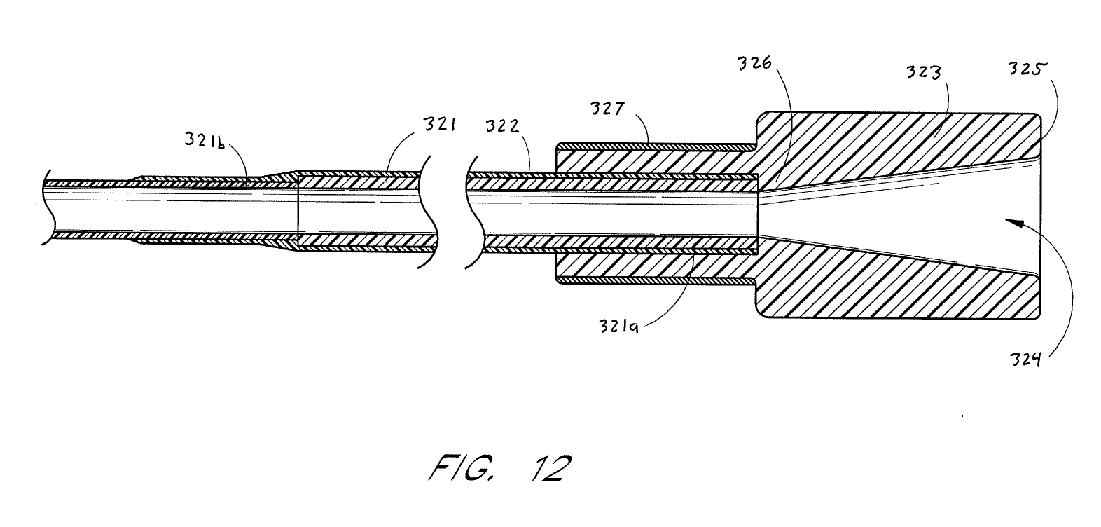

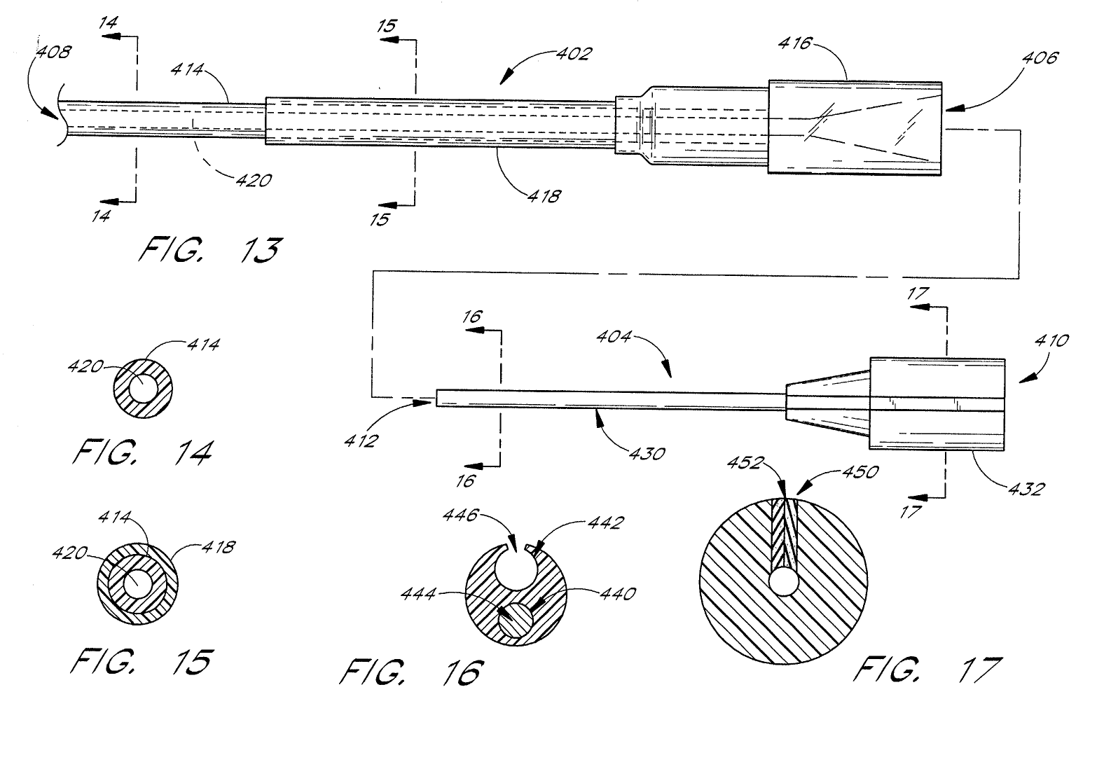

It will be noted that various configurations and dimensions are possible with respect to the proximal portion 14b in order to accommodate the balloon or other medical device intended for use in connection with the present protective sheath assembly. Furthermore, the combination of a larger diameter distal section and a smaller diameter proximal section may be reversed, depending upon the configuration of the catheter or other medical device to be protected by the sheath assembly. Thus, as discussed below in more detail in connection with FIGS. 6 and 10, other combinations of lumen configurations are within the scope of the present invention. However, one preferred application of the present sheath assembly is illustrated in FIG. 4. FIG. 4 illustrates a broken side cross-sectional view of the sheath assembly of FIG. 1 and further illustrates in dotted lines a catheter 15 positioned within the lumen 14 of the protective sheath 1. Specifically, the catheter 15 comprises a guidewire 17 extending from the proximal end 20 of the sheath 1 and toward the distal end 10. Mounted on the distal end of the guidewire 17 is a medical balloon 19 which is housed protectively within the distal portion 14a of the sheath 1. It will be noted that the guidewire 17 is housed snugly in the proximal portion 14b of the lumen of the sheath in order to prevent or at least minimize back blood flow under arterial pressure. It will be noted that the longitudinal position of the balloon is not particularly important so long as it is protectively contained within the lumen 14a. In a preferred method, the proximal end of the guidewire 17 is loaded into the sheath 1 beginning at the distal end 10. This loading is facilitated by a transition section 21, as illustrated in FIG. 4, located between the distal section 14a and the proximal section 14b of the lumen 14. This lumen transition 21 between the proximal portion 14a and the distal portion 14b should be smooth to assist the loading of a balloon guidewire. Although, as noted above, the present chief assembly can be utilized with a wide variety of medical devices, one fixed wire catheter which is suitable for use therewith is illustrated and described in detail in U.S. Pat. Nos. 6,068,623, 6,050,972 and 5,868,705, as well as application Ser. No. 09/653,218, filed Aug. 31, 2000, the entirety of which are hereby incorporated by reference.

Referring again to FIG. 1, the female luer lock 3 has a cup-shaped cavity 30 at its proximal side, a cylindrical cavity 32 at its distal side, a through-hole 34 in the center along its longitudinal axis, and a wing 36. The strain-relief tubing 2 has an inner diameter substantially the same as, or slightly larger than the outer diameter of the proximal portion of the lumen 14 and forms a tight fit with the protective sheath 1. At the distal end of the strain-relief tubing 2, its inner surface is flared to fit the outer surface of the protective sheath 1 as shown in FIG. 4. The outer diameter of the strain-relief tubing matches with the inner diameter of the cylindrical cavity 32 so that they can be connected by slip-fit. The insertion of the proximal portion of the lumen 14 into the strain-relief tubing 2 is also accomplished by slip-fitting. The cup-shaped cavity 30 serves to optionally receive a needle introducer (not shown), or flushing devices.

The particular protective sheath assembly, shown as an example in FIG. 1, has the following dimensions: the total length of the protective sheath 1 is about 3.6 in. with the proximal portion about 1 in., the distal portion about 2.5 in., and the transition between these two portions about 0.1 in. The inner diameter of the proximal portion is about 0.017 in., the inner diameter of the distal portion is about 0.05 to 0.075 inch. The length of the strain-relief tubing 2 is about 1 inch. However, it will be appreciated that the protective sheath assembly is not limited to any specific dimensions.

In FIG. 6, another embodiment is shown. The protective sheath assembly has a protective sheath 101, a strain-relief tubing 102, and a female luer lock 103 similar to those discussed above in FIG. 1. However, in this embodiment a metallic hypotube (or a needle) 40 is inserted into the proximal end 20 of the protective sheath 101. The outer diameter of the metallic hypotube is slightly larger than the original inner diameter of the proximal portion of the lumen 114. The metallic hypotube is inserted into the smaller diameter of the lumen 114, preferably by heating the protective sheath to a temperature high enough to soften it. The metallic hypotube is forced into the proximal portion of the lumen 114, followed by cooling and subsequent hardening of the protective sheath. By this heating and cooling process, the hypotube 40 is firmly held within the proximal portion 20 of the protective sheath 101. If necessary, the heating and cooling process can be conducted after the strain-relief 102 tubing has been inserted onto the proximal portion 20 of the protective sheath 101. The insertion of the hypotube 40 divides the proximal portion of the protective sheath 101 into two parts: a first part 42 encloses the hypotube 40, and a second part 44 which is not in contact with the metallic hypotube 40. As shown in FIG. 6, the inner diameter of the proximal portion second part 44, which is defined by the original inner diameter of the proximal portion of the protective sheath 101, is smaller than the inner diameter of the hypotube 40. A larger inner diameter of the hypotube 40 facilitates smooth movement of a guidewire, while the second part 44 provides a good seal around the guidewire. Thus, by adjusting the relative length of the hypotube 40 and the second part 44 one may balance the requirements of attaining smooth guidewire movement as well as a good seal around the guidewire. The distal end of the protective sheath 101 may have a flared inner diameter and a tapered outer diameter, as illustrated in the embodiment of FIG. 1.

The protective sheath, the strain-relief tubing, and the hub may be molded into a single piece. Although the protective sheath assembly of FIG. 6 is not limited to any specific dimension, this embodiment has the following dimensions: the protective sheath 101 has total length about 10-12.5 cm with the first part 42 about 3-4 cm, the second part 44 about 1-1.5 cm (including the smooth transition part) and the distal portion about 6-7 cm. The inner and outer diameter of the distal portion of the protective sheath 101 are 0.05 to 0.075 in. and 0.08 to 0.10 in., respectively. The inner diameter of the second part 44 is about 0.017 in. The inner and outer diameter of the metallic hypotube 40 are about 0.02 and 0.035 in., respectively. The strain-relief tubing 102 has a length about 4-5.5 cm.

The protective sheath, the female luer lock, and the strain-relief tubing can be made from various polymer materials such as PEBAX, PE, PEEK, FEP, PTFE, polyimide (Nylon), polycarbonate, a silicone-based material, etc. Additionally, different parts of the same component may be constructed from different materials. For instance, in any of the preferred embodiments described herein, the distal end of the protective sheath may be constructed from a relatively stiff material (e.g., PEBAX), whereas the proximal end is constructed from a more flexible material (e.g., a silicone-based material). Alternatively, the proximal end may be constructed from a stiffer material and the distal end constructed from a more flexible material. Varying the flexibility of the protective sheath along its length allows for easier manipulation of the protective sheath assembly at its relatively more flexible portions, while still providing stability at its relatively stiffer portions. Additionally, constructing the portion of the protective sheath assembly that comes into direct contact with the hemostasis valve from a flexible material creates a tighter fit around the catheter within the protective sheath, thereby further minimizing blood backflow. The female luer lock or connecting member can also be made of metals such as stainless steel.

As shown in FIG. 12, in a preferred embodiment, the protective sheath assembly 320 comprises a protective sheath 321 having a proximal end 321a and a distal end 321b, a strain-relief tubing 322, and a connecting member 323. The distal end 321b of the protective sheath 321 and the strain-relief tubing 322 are constructed from PEBAX. The proximal end 321a is constructed from a flexible, silicone-based material. The connecting member 323 is constructed from polycarbonate. Additionally, the through-hole 324 of the connecting member 323 tapers uniformly from its proximal end 325 to its distal end 326.

There are various ways to mount different parts of the protective sheath assembly. For example, the strain-relief tubing and the protective sheath can be glued together, or produced as a single unit, or as shrink tubing (which is shrunk). The strain-relief tubing can be mounted to the female luer lock through a ridge-grooved mechanism. The female luer lock can be replaced with other connecting devices. Furthermore, as shown in FIG. 12, tubing 327 may be heat-shrunk over the connecting member 323 (shown) and/or the strain-relief tubing 322 (not shown) for additional bond strength. The tubing 324 preferably is made from FEP. These variations would be obvious to one skilled in the art and are considered within the scope and spirit of the present invention.

Yet another embodiment of the sheath assembly is shown in FIGS. 10A and 10B. This embodiment may be constructed at its proximal end 120 in a manner similar to either of the embodiments of FIG. 1 or 6. However, the distal end of the sheath 105, on the other hand, is provided with multiple cross-sectional diameters. Thus, in this embodiment, a larger diameter section is shown at the distal end of the sheath with a conical or frusto-conical transition section 119 causing a lumen 115 to transition down to a smaller diameter section 121. This smaller section in turn is further reduced down through another conical section 123 to form the smallest diameter 125, which passes through the proximal end to provide a sealing portion around the guidewire. The sheath of this embodiment helps to accommodate catheters having similar transitions in diameter, or it can accommodate different balloon sizes.

Construction and Use

In addition to the methods of construction discussed above in connection with FIG. 6, methods for making the protective sheath and protective sheath assembly are provided. The protective sheath can be made by necking down a polymer tubing of an appropriate size, bonding a strain-relief tubing or sleeve over the proximal portion of the protective sheath, flaring the inner surface, tapering the outer surface at the distal end of the protective sheath, and attaching a hub or connecting member to the proximal end of the protective sheath. The sheath can also be made from a variable diameter extruded plastic without the need for necking.

In one embodiment, the method for making the protective sheath comprises the following steps: (1) extruding a plastic tubing such as a PE tubing of desired diameter and length; (2) inserting a mandrel of desired outer diameter into the plastic tubing; (3) exposing 1.5-2 in. of the plastic tubing at one end in a hot air box and covering the remaining portion of the plastic tubing with a PTEE sleeve; (4) heating and stretching the exposed end of the plastic tubing in the hot air box; (5) flaring the necked end by inserting a tapered pin at an appropriate temperature in the air box and trimming the ends. Also, it is apparent that various modifications can be made in the above discussed process. Once the protective sheath is made, it can be easily assembled with a connecting member and a strain-relief tubing, or with an introducer. It is also possible to extrude a tube with variable diameter.

In constructing any of the preferred embodiments of the protective sheath assembly described herein, the lumen of the protective sheath may be coated with a silicone coating to facilitate movement of the catheter therethrough. Additionally, the through-hole of the connecting member may be coated with a silicone coating.

Method of Use

Referring now to FIG. 11, in using the protective sheath assembly or the introducer/protective sheath assembly, a guide catheter is first inserted into the blood vessel according to the procedure described in the background section, and incorporated herein. As shown in FIG. 11, the guide catheter 300 is inserted into a blood vessel 302 through an optional arterial sheath 304. The arterial sheath 304 is inserted into the blood vessel through the skin 306 according to steps (a)-(c) described in the background section. A Y-adaptor 308 is connected to the proximal end of the guiding catheter 300. A hemostasis valve or a Touhy-Borst valve is installed inside the Y-adaptor to prevent blood flow. A DOW (or other fixed-wire catheter) 312 is inserted into the protective sheath assembly 310 by introducing the proximal end of the DOW 312 (the end without a balloon) into the distal end of the protective sheath assembly 310 and advancing it until the balloon and the soft tip at the distal end of the DOW are accommodated inside the proximal portion of the protective sheath assembly 310. The protective sheath assembly is then inserted into the Y-adaptor 308 with the distal end of the protective sheath assembly 310 passing the hemostasis valve mounted in the Y-adaptor 308. The DOW is then advanced toward the treatment site, followed by removal of the protective sheath assembly 310. The removal of the protective sheath assembly also can be done before the DOW 312 is advanced toward the treatment site. In this way, a balloon catheter with a soft tip can be easily and quickly loaded into a blood vessel through the protective sheath assembly, via introduction through a Y-adaptor and a hemostasis valve.

Additional Embodiments for Use with Filter Devices

Additional embodiments of the protective sheath assembly are designed for use with various intravascular occlusion devices, such as, for example, filter devices. A filter device generally comprises a radially expanding filter located at the distal end of an elongated filter shaft (or wire). The expandable filter is preferably formed from a plurality of struts connected to the filter shaft, with a flexible material or membrane attached to the struts having pores sized for trapping emboli in the blood. The expandable filter is typically a delicate structure that can easily be damaged during insertion through a hemostasis valve (or other similar valve) into the vasculature of a patient.

One example of a filter device is described with respect to FIGS. 56-66 below. Further details regarding vascular filter devices are disclosed in Applicant's copending applications entitled: 1) MEMBRANES FOR MECHANICAL OCCLUSION DEVICE AND METHODS AND APPARATUS FOR REDUCING CLOGGING, Ser. No. 09/505,554, filed Feb. 17, 2000; 2) STRUT DESIGN FOR AN OCCLUSION DEVICE, Ser. No. 09/505,546, filed Feb. 17, 2000; and 3) OCCLUSION OF A VESSEL AND ADAPTOR THEREFOR, Ser. No. 09/505,911, filed Feb. 17, 2000, the entirety of each of which is hereby incorporated by reference.

During intravascular treatment, a hemostasis valve is typically located proximal to the opening of a guide catheter and is connected to the guide catheter through a luer lock. An intravascular device is inserted into a patient's vasculature through the hemostasis valve. The hemostasis valve is configured with slits and/or holes that are normally closed, but are adapted to open sufficiently wide such that the intravascular device can be inserted through the valve. The hemostasis valve provides a seal around the intrasvascular device to prevent blood back flow through the valve at arterial pressure. However, when a delicate structure, such as an expandable filter device, is inserted through a hemostasis valve, the structure may become damaged due to snagging or friction with the valve.

Therefore, a need exists for a protective sheath assembly that can protect intravascular devices from becoming damaged during insertion through a hemostasis valve. To be practical, the protective sheath must maintain a tight seal around the device to prevent blood back flow while allowing for smooth advancement of the device therethrough. The protective sheath should be reliable, strong and capable of being adapted for use with a wide variety of intravascular devices.

In response to this need, a protective sheath assembly is provided that is adapted for receiving an intravascular device. The protective sheath protects the intravascular device against damage as the device is passed through an obstruction or valve, such as a hemostasis valve. Various embodiments are described herein with respect to a particular application. The application involves a type of filter device that is protected during insertion through a hemostasis valve. However, it will be appreciated by those with ordinary skill in the art that the protective sheath may also be advantageously used with other types of filter devices, as well as other occlusion devices utilizing balloons.

Referring now to FIG. 13, for purposes of illustration, a first embodiment of a protective sheath assembly adapted for use with a filter device generally comprises an outer sheath 402 and an inner sheath 404. The outer sheath 402 is similar to the protective sheath assembly adapted for use with a balloon catheter as described above with reference to FIG. 1, however, this embodiment does not include a female luer lock. The outer sheath 402 is provided with a proximal end 406 and distal end 408 and generally comprises an elongated tubular body 414, a proximal hub 416, and a strain-relief tubing 418. A cross-sectional view of the distal portion of the elongated tubular body 414 is shown in FIG. 14 in which the elongated tubular body 414 defines the lumen 420. A cross-sectional view of the proximal portion of the elongated tubular body 414 is shown in FIG. 15 in which a strain relief tubing 418 surrounds the elongated tubular body 414.

Referring again to FIG. 13, the inner sheath 404 is provided with a proximal end 410 and a distal end 412 and generally comprises an elongated inner body 430 and a proximal hub 432. As shown in FIG. 16, the elongated inner body 430 is provided with a shaft lumen 442 for accommodating the shaft (or wire) of the filter device. The diameter of the shaft lumen 442 is slightly larger than the outer diameter of the filter shaft (not shown), thereby allowing for smooth advancement of the filter shaft through the inner sheath 404 while minimizing blood back flow between the shaft lumen 442 and the filter shaft. With the filter shaft inserted into the shaft lumen 442 of the inner sheath 404, the elongated inner body 430 of the inner sheath 404 is inserted into the elongated lumen 420 of the outer sheath 402. Referring again to FIG. 13, the outer diameter of the elongated inner body 430 of the inner sheath 404 and the diameter of the elongated lumen 420 in the outer sheath 402 are roughly equivalent. Therefore, when the inner sheath 404 is inserted into the outer sheath 402, the inner sheath 404 creates a tight fit in the elongated lumen 420 of the outer sheath 402 and provides a seal against blood back flow.

Referring again to FIG. 16, the elongated inner body 430 of the inner sheath 404 is preferably provided with a shaft lumen 442 and a mandrel lumen 440. The mandrel lumen 440 contains a mandrel 444 for providing structural support to the inner sheath 404. However, if the elongated inner body 430 is formed of a sufficiently strong material such as PEEK, the mandrel 444 and mandrel lumen 440 may not be necessary. The shaft lumen 442 extends the entire length of the elongated inner body 430 and provides the passageway for the filter shaft (or wire) to pass through the device. The shaft lumen 442 is formed with a longitudinal slot 446 that makes it possible to side load the filter shaft into the shaft lumen 442 by snapping the filter shaft through the longitudinal slot 446 into the shaft lumen 442. The shaft lumen 442 is formed with smooth walls so that the filter shaft may be easily and smoothly advanced through the device.

The inner sheath 404 includes a proximal hub 432 for gripping and retrieving the inner sheath 404. As shown in FIG. 17, the proximal hub 432 is formed with a hub slot 450 containing two compressible pads 452. The compressible pads 452 allow the filter shaft to be passed through the hub slot 450 into the lumen of the proximal hub 432 by applying sufficient force. The proximal hub 432 of the inner sheath 404 is formed to mate with the hub 416 of the outer sheath 402 when the inner sheath 404 is fully inserted into the elongated lumen 420 of the outer sheath 402.

The inner and outer sheaths are preferably constructed from a polymer material such as PEBAX, PE, PEEK, FEP, PTFE, polyimide (Nylon), or polycarbonate. The outer sheath is designed to accommodate the expandable filter and preferably has a lumen with an inner diameter ranging from about 0.045 inches to 0.065 inches. The inner sheath 404 is designed to accommodate the filter shaft. The filter shaft used with one embodiment has a diameter of about 0.014 inches. The inner sheath 404 has a shaft lumen 442 with an inner diameter preferably ranging from about 0.016 to 0.020 inches.

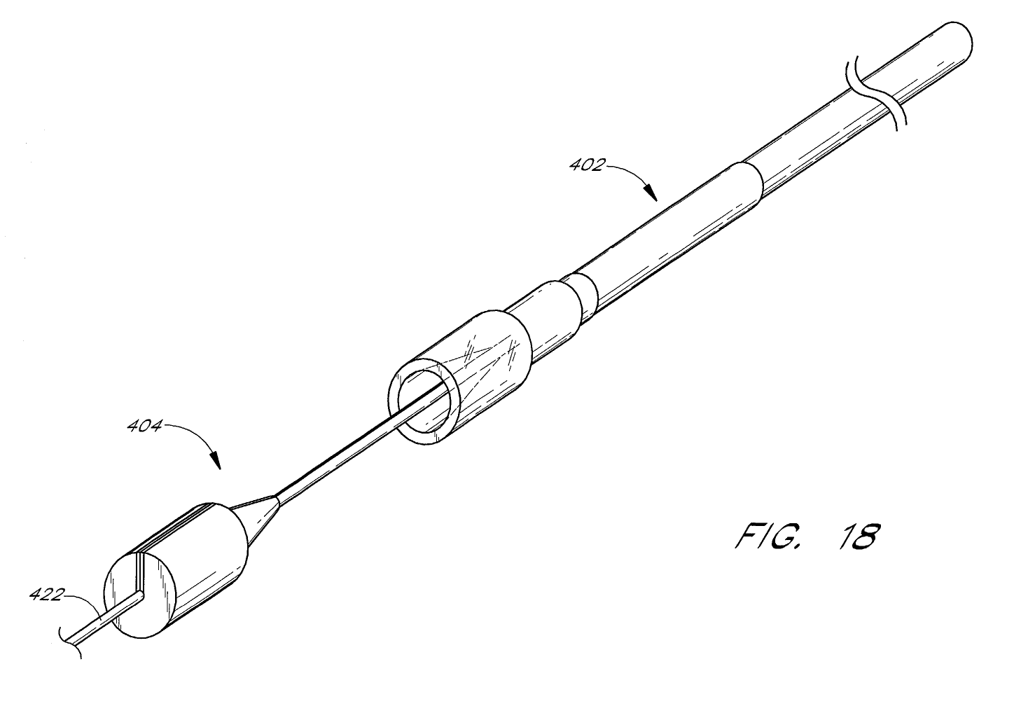

In use, the inner sheath 404 is first removed from the elongated lumen 420 of the outer sheath 402. The filter device is then front loaded or back loaded into the lumen 420 of the outer sheath 402 until the expandable filter is contained within the distal portion of the elongated lumen 420 and the filter shaft is extending out of the proximal end 406 of the outer sheath 402. The filter shaft is then side-loaded into the shaft lumen 442 of the inner sheath 404 by snapping the filter shaft through the longitudinal slot 446 and pushing the filter shaft between the compressible pads 452 on the proximal hub 432. With the filter shaft contained within the shaft lumen 442 of the inner sheath 404, the inner sheath 404 is then inserted into the elongated lumen 420 of the outer sheath 402. FIG. 18 illustrates a configuration wherein the filter shaft 422 is contained within the shaft lumen of the inner sheath 404 and the inner sheath 404 is partially inserted into the elongated lumen 420 of the outer sheath 402. The protective sheath assembly is then inserted through the hemostasis valve and the filter device can be safely advanced through the hemostasis valve into the vasculature of the patient.

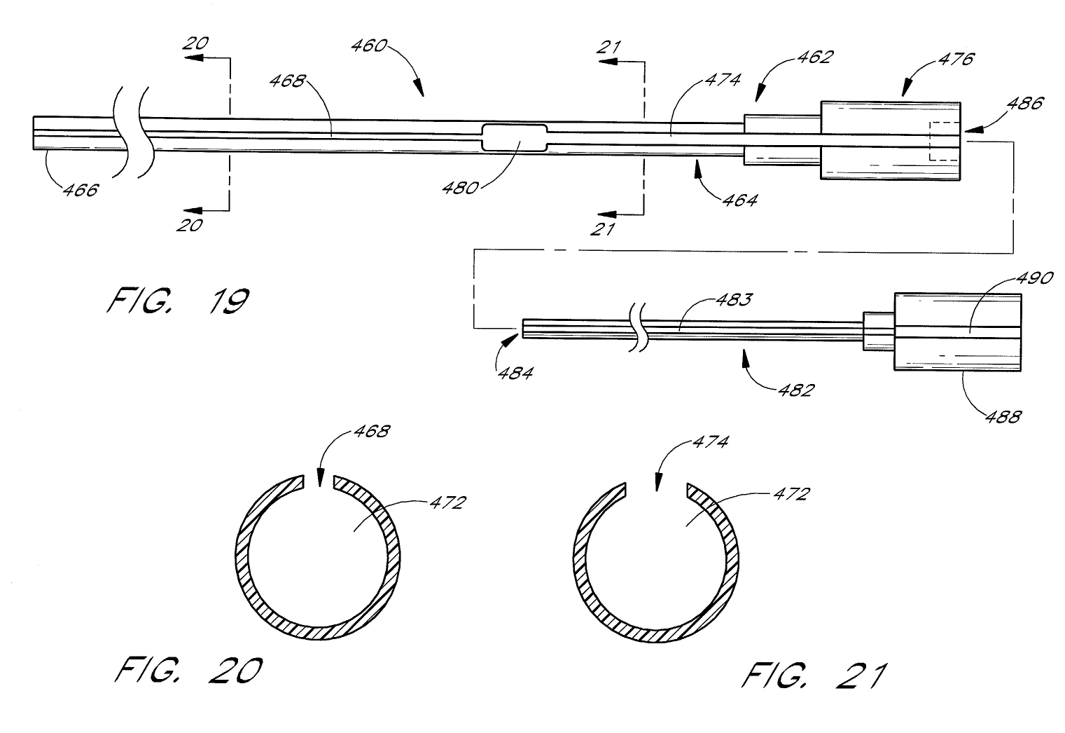

A second embodiment of a protective sheath assembly adapted for use with a filter device provides an outer sheath 460 formed with two longitudinal slots 468, 474. This embodiment allows the filter device to be completely side-loaded into the protective sheath while the inner sheath 482 is inserted into the outer sheath 460. As illustrated in FIG. 19, the elongated tubular body 462 of the outer sheath 460 is formed with a proximal portion 464, a distal portion 466 and an entrance port 480. The proximal portion 464 of the elongated tubular body 462 is formed with a proximal slot 474 extending along the longitudinal axis from the proximal end 486 to the entrance port 480. FIG. 21 shows a cross-sectional view of the proximal portion 464 of the elongated tubular body 462 having a proximal slot 474. The width of the proximal slot 474 is formed slightly smaller than the diameter of the filter shaft thereby allowing the filter shaft to be snapped through the proximal slot 474 by applying a small amount of force.

The distal portion 466 of the elongated tubular body 462 is formed with a distal slot 468 extending along the longitudinal axis from the entrance port 480 to the distal end of the elongated tubular body 462. The width of the distal slot 468 is much smaller than the diameter of the filter shaft. However, the distal portion 466 of the elongated tubular body 462 is manufactured using a pliable material that will yield sufficiently to allow the filter shaft to be peeled out of the shaft lumen through the distal slot 468. FIG. 20 shows a cross-sectional view of the distal portion 466 of the elongated tubular body 462 having a distal slot 468 and defining a lumen 472.

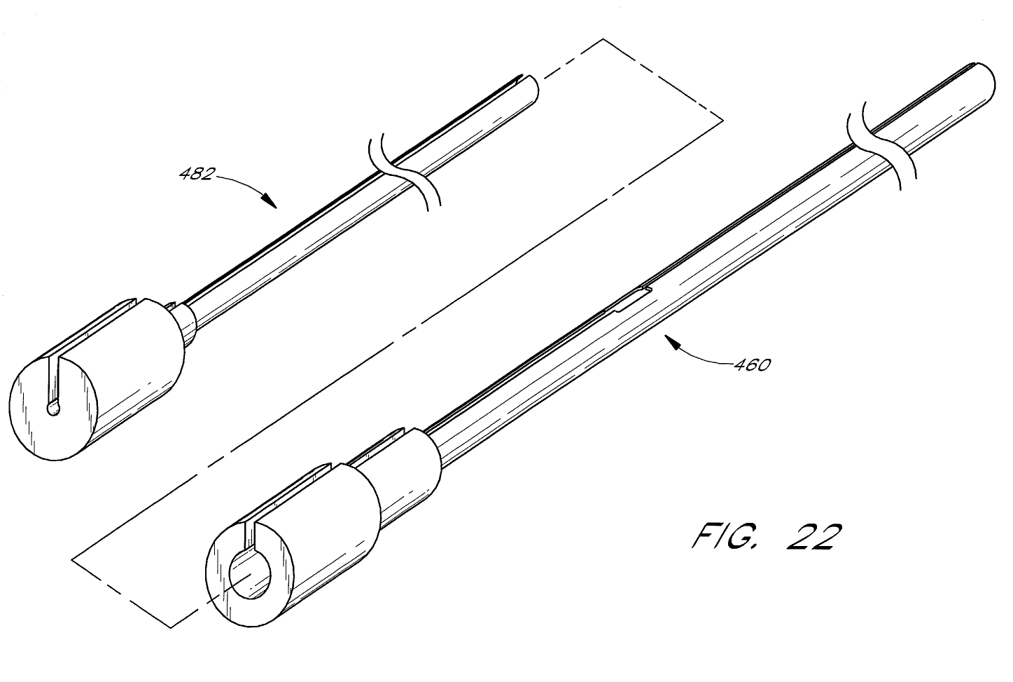

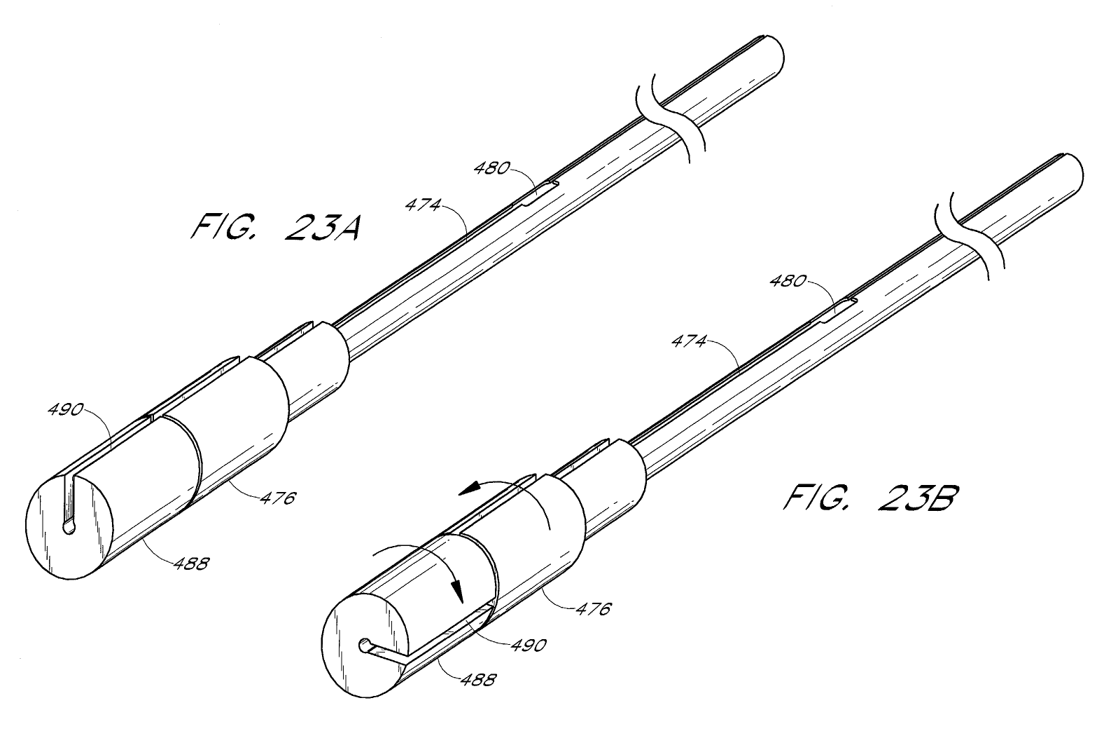

Referring again to FIG. 19, the inner sheath 482 has a distal end 484 that is inserted into the lumen 472 of the outer sheath 460 through the proximal end 486. Preferably, when fully inserted, the distal end 484 of the inner sheath 482 extends to a point just proximal of the entrance port 480, however, the length of the inner sheath 482 may vary. The inner sheath 482 includes a shaft lumen (not shown) and has a proximal hub 488 at its proximal end that is formed with a hub slot 490. The inner sheath may also include a mandrel lumen for structural support as described above. The inner sheath 482 can be rotated relative to the outer sheath 460 to move the slot 483 in the inner sheath 482 into or out of alignment with the proximal slot 474 in the outer sheath 460. FIG. 22 shows a perspective view of the inner sheath 482 and outer sheath 460 with the inner sheath 482 removed from the lumen 472 of the outer sheath 460.

In use, the inner sheath 482 is initially inserted into the lumen 472 of the outer sheath 460 such that the proximal slot 474 in the outer sheath 460 is aligned with the slot in the inner sheath 482 as shown in FIG. 23A. In this configuration, the expandable filter is inserted through the entrance port 480 in the outer sheath 460 and is advanced into the distal portion 466 of the lumen 472 of the outer sheath 460. Next, the filter shaft (or wire) is snapped through the proximal slot 474 in the proximal portion 464 of the outer sheath 460. Because the slot 483 in the inner sheath 482 is aligned with the proximal slot 474 in the outer sheath 460, the filter shaft may also be snapped into the shaft lumen 487 of the inner sheath 482 in the same continuous motion. With the filter shaft contained within the shaft lumen of the inner sheath 482, the inner sheath 482 is then rotated relative to the outer sheath 460 such that the slots are no longer in alignment as shown in FIG. 23B.

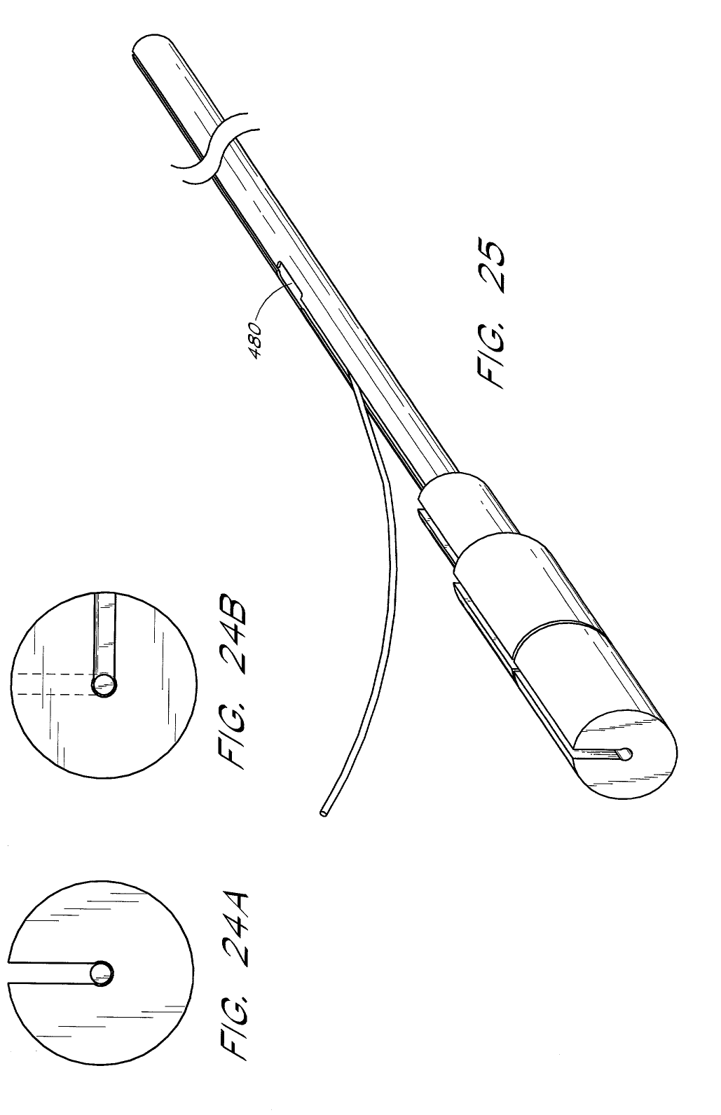

In this configuration, the filter shaft cannot be removed from the shaft lumen 487 of the inner sheath 482. This feature provides a locking mechanism to prevent the filter shaft from inadvertently coming out of the protective sheath assembly. FIG. 24A shows a back end view of this embodiment with the slots in alignment such that the filter shaft may be loaded or removed from the shaft lumen 487 of the inner sheath 482. FIG. 24B shows a back end view of this embodiment with the slots out of alignment such that the filter shaft is locked in the shaft lumen 487 of the inner sheath 482. With the filter shaft locked in the shaft lumen 487, the protective sheath assembly is then inserted through the hemostasis valve and the filter device is advanced into the vasculature of the patient. After the expandable filter has been safely advanced through the hemostasis valve, the protective sheath assembly is backed out of the valve and the slots are realigned to remove the protective sheath from the filter device by peeling the filter shaft through the slots as shown in FIG. 25.

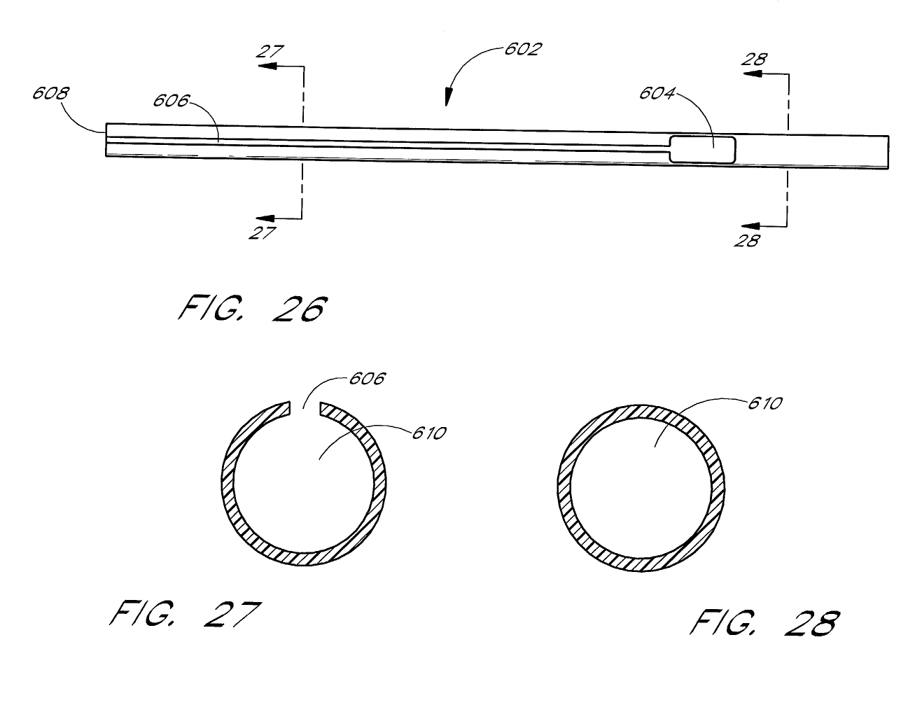

A third embodiment of a protective sheath assembly adapted for use with a filter device provides a single protective filter sheath 602 that can be used in combination with a wide variety of existing wire introducers. A wire introducer typically includes a long tubular body defining a lumen that is slightly larger than the diameter of the filter shaft (or wire). Referring now to FIG. 26, a single filter sheath 602 is disclosed comprising an elongated tubular body 603 defining a lumen 610 having a diameter slightly larger than the diameter of the expandable filter. The filter sheath is formed with an entrance port 604 and a single slot 606 extending longitudinally from the entrance port to the distal end 608. FIG. 27 shows a cross-section of the distal portion of the filter sheath 602 having a slot 606 and defining a lumen 610. FIG. 28 shows a cross-section of the proximal portion of the filter sheath 602 comprising a solid hollow tube defining a lumen 610.

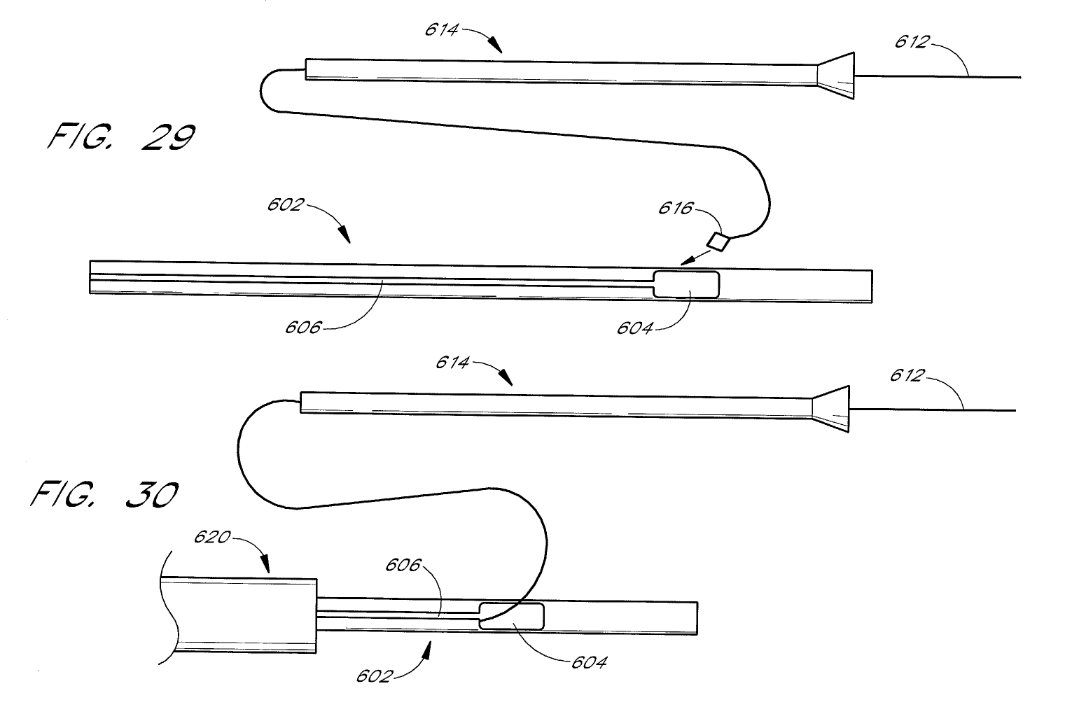

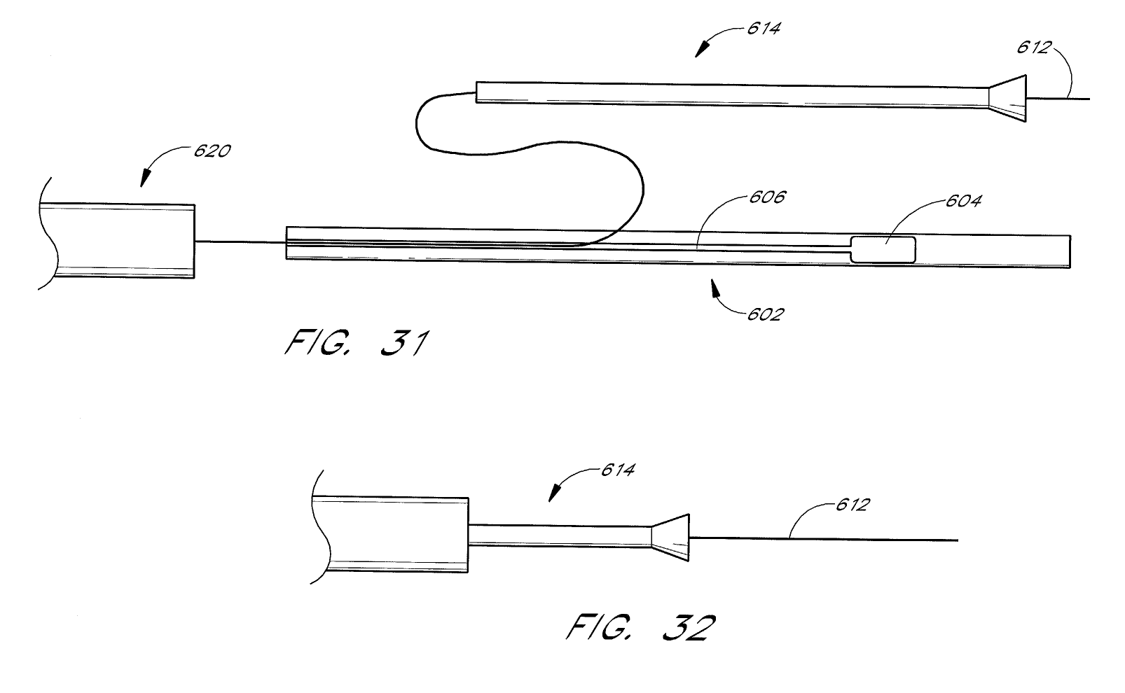

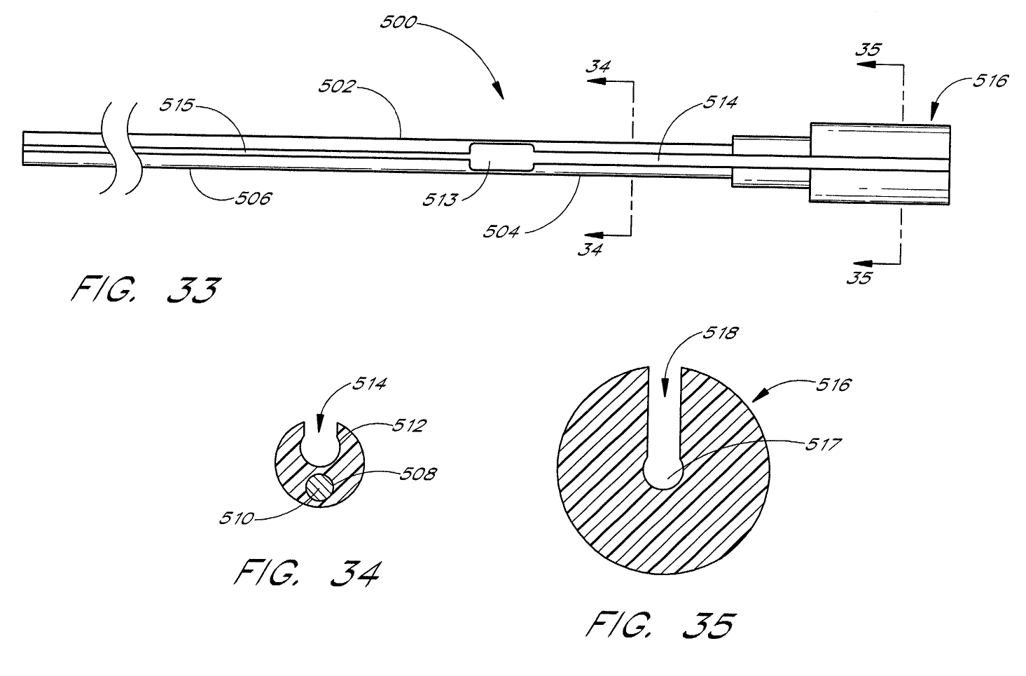

FIGS. 29-32 illustrate a preferred use of the embodiment just described with reference to FIGS. 26-28. As shown in FIG. 29, the shaft 612 of the filter device is first back loaded into the lumen of the wire introducer 614. The expandable filter 616 is then inserted through the entrance port 604 in the side of the filter sheath 602 and is advanced into the distal portion of the filter sheath 602. As shown in FIG. 30, the distal portion of the filter sheath 602 is then inserted through the hemostasis valve 620 so that the expandable filter can be safely advanced past the hemostasis valve 620 through the lumen 610 of the filter sheath 602. As shown in FIG. 31, the filter sheath 602 is then backed out of the hemostasis valve 620 and the filter shaft 612 is peeled through the slot 604 to remove the filter sheath 602 . The wire introducer 614 is then inserted into the hemostasis valve 620 to provide a seal against blood back flow and to facilitate movement of the filter shaft 612 through the valve 620 as shown in FIG. 32. The wire introducer 612 allows the valve 620 to be tightened down to minimize blood back flow without affecting the ability to advance the filter shaft 612 through the valve 620.

A fourth embodiment of a protective sheath assembly adapted for use with a filter device is disclosed in FIGS. 33-36. This embodiment provides a single-piece protective sheath assembly generally comprising an elongated tubular body 502 having a proximal portion 504, a distal portion 506 and a proximal hub 516. The proximal portion defines a shaft lumen for accommodating the filter shaft and the distal portion defines a larger lumen (not shown) for accommodating the expandable filter. The proximal hub 516 is connected to the proximal end of the elongated tubular body 502 for gripping and retrieving the protective sheath assembly 500. As shown in FIG. 35, the proximal hub 516 is formed with a slot 518 for providing access to the lumen 517. The slot 518 may contain compressible pads (not shown) to help maintain the filter shaft in the lumen 517.

Referring now to FIGS. 33-34, the proximal portion 504 of the protective sheath assembly is formed with a shaft lumen 512 having a diameter slightly larger than the filter shaft thereby allowing for advancement of the filter shaft through the shaft lumen 512 while still minimizing blood back flow. Preferably, the proximal portion 504 of the elongated tubular body 502 also includes a mandrel lumen 508 containing a mandrel 510 for structural support. A slot 514 is formed on the side of the proximal portion 504 thereby allowing the filter shaft to be side loaded into the shaft lumen 512.

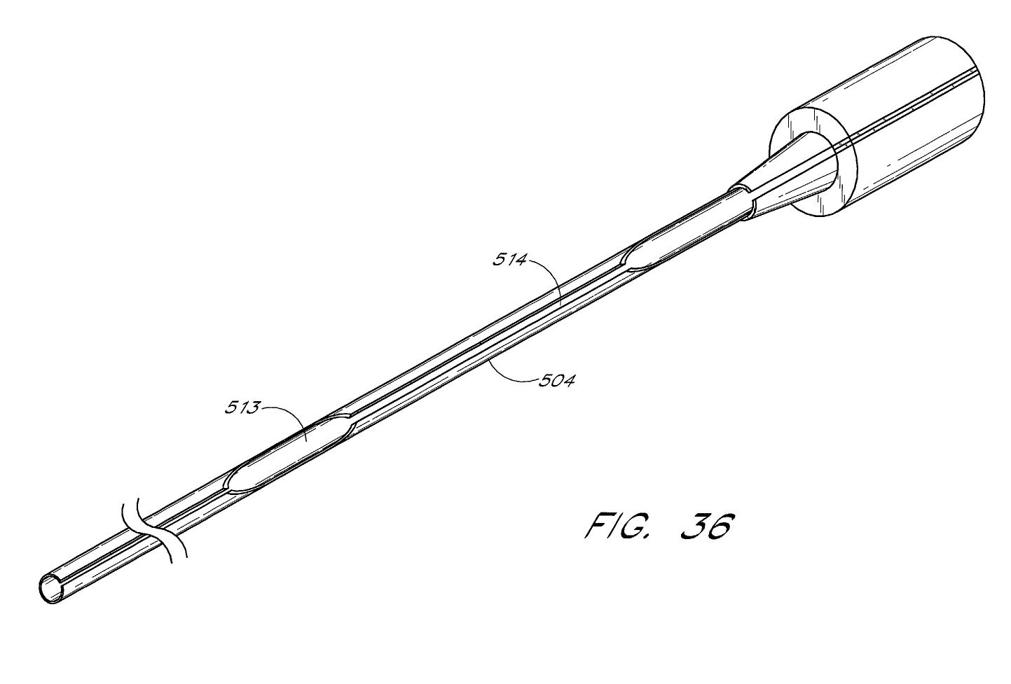

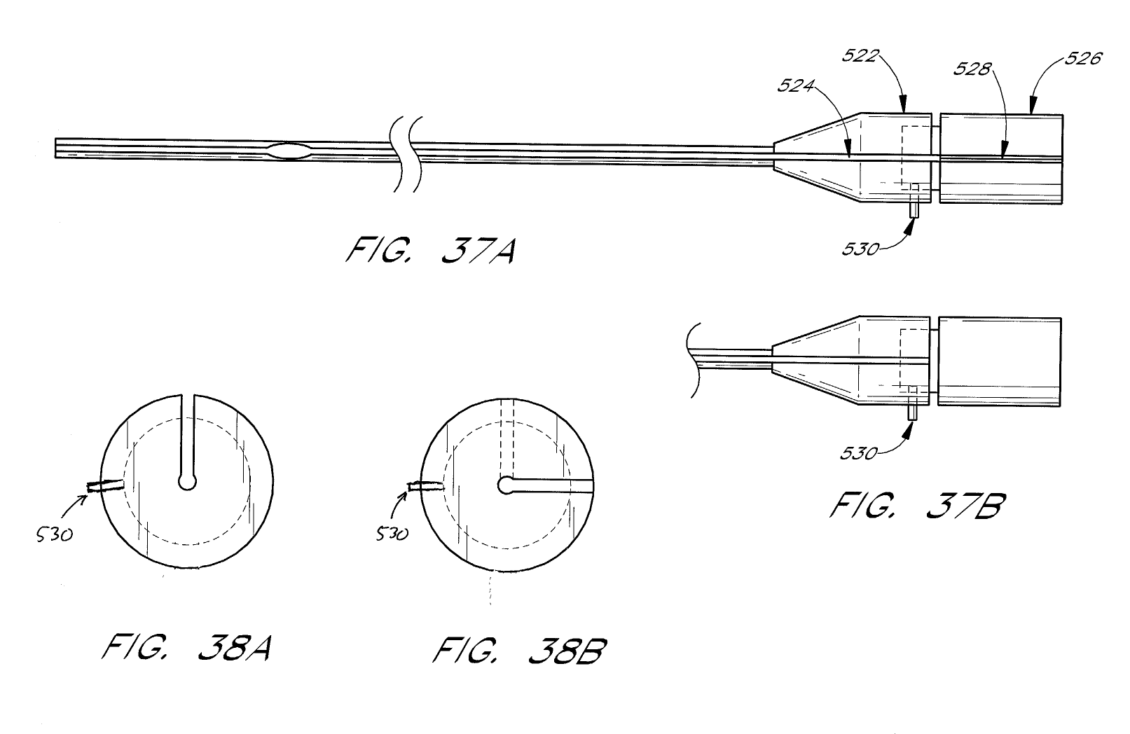

The distal portion 506 of the protective sheath assembly includes a single lumen (not shown) with a diameter designed to accommodate the expandable filter. A distal slot 515 is formed in the distal portion 506 which allows the filter shaft to be peeled out of the protective sheath 500 after the filter has been advanced through the valve into the vasculature of the patient. FIG. 36 shows a perspective view of this embodiment.

In use, the expandable filter at the distal end of the filter device is inserted through the entrance port 513 into the distal portion 506 of the elongated tubular body 502. The filter shaft is then snapped through the proximal slot 514 into the shaft lumen 512. The filter shaft is also inserted through the hub slot 518 into lumen 517 at the center of the hub 516. The protective sheath assembly 500 is then inserted into the hemostasis valve and the expandable filter is advanced into the blood vessel. Once the expandable filter has been safely advanced past the hemostasis valve, the protective sheath assembly 500 may be backed out of the valve and the filter shaft may then be peeled through the slots on the side of the protective sheath assembly 500. The protective sheath assembly 500 may then be completely removed from the filter device and thrown away or reused during a subsequent procedure.

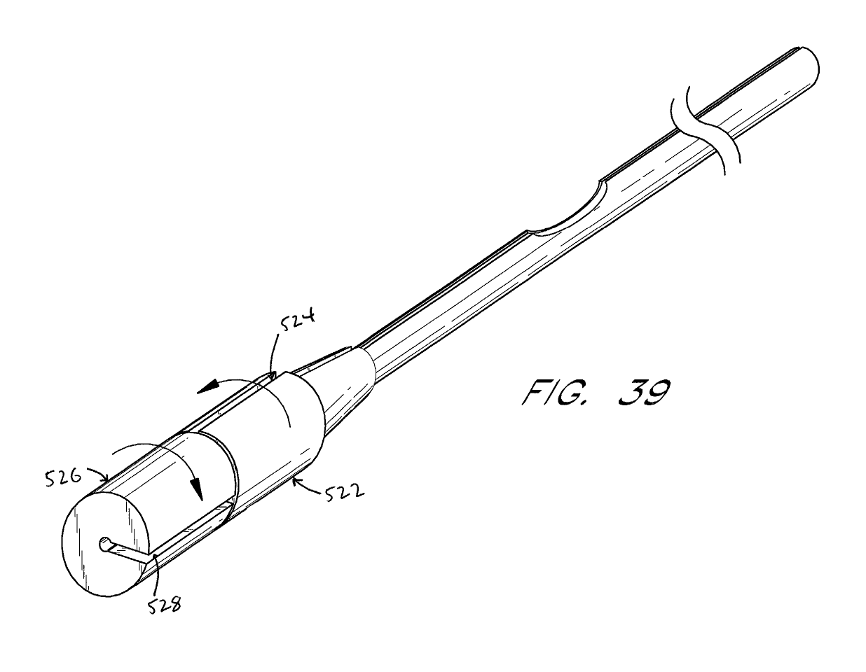

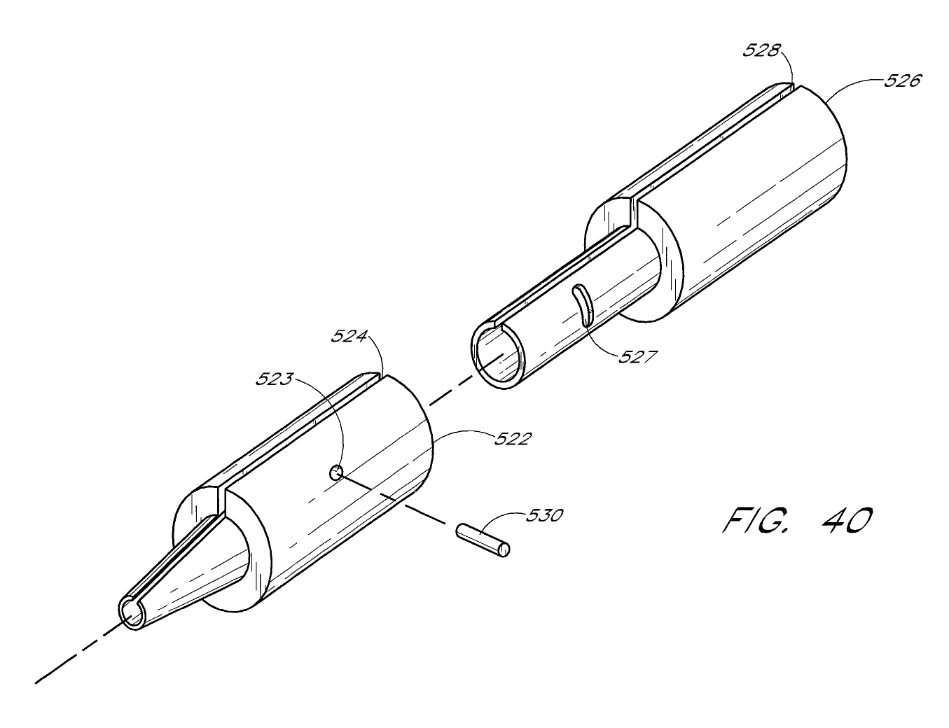

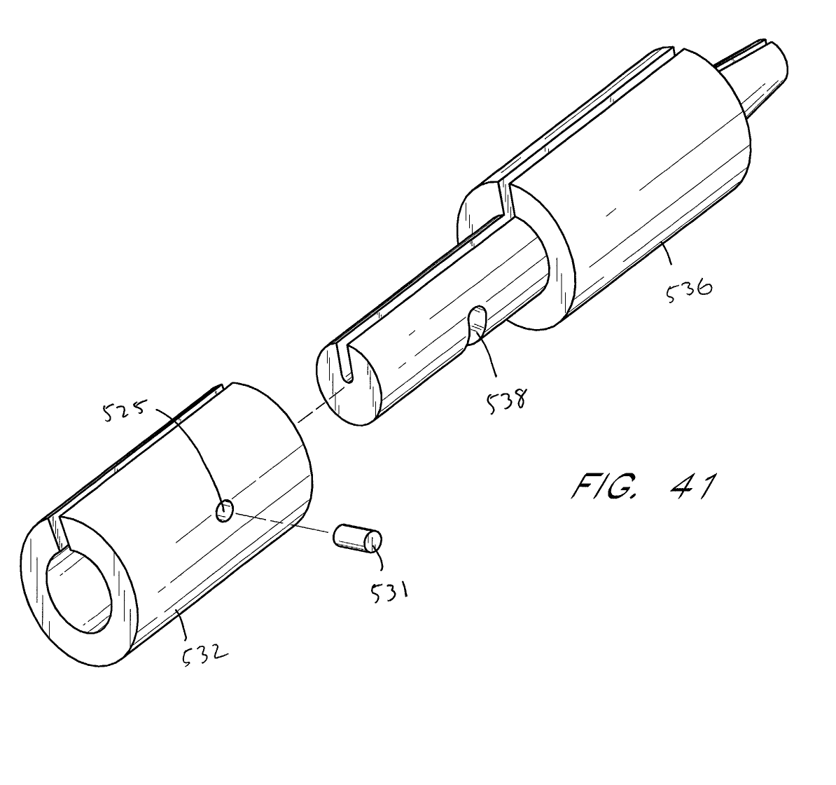

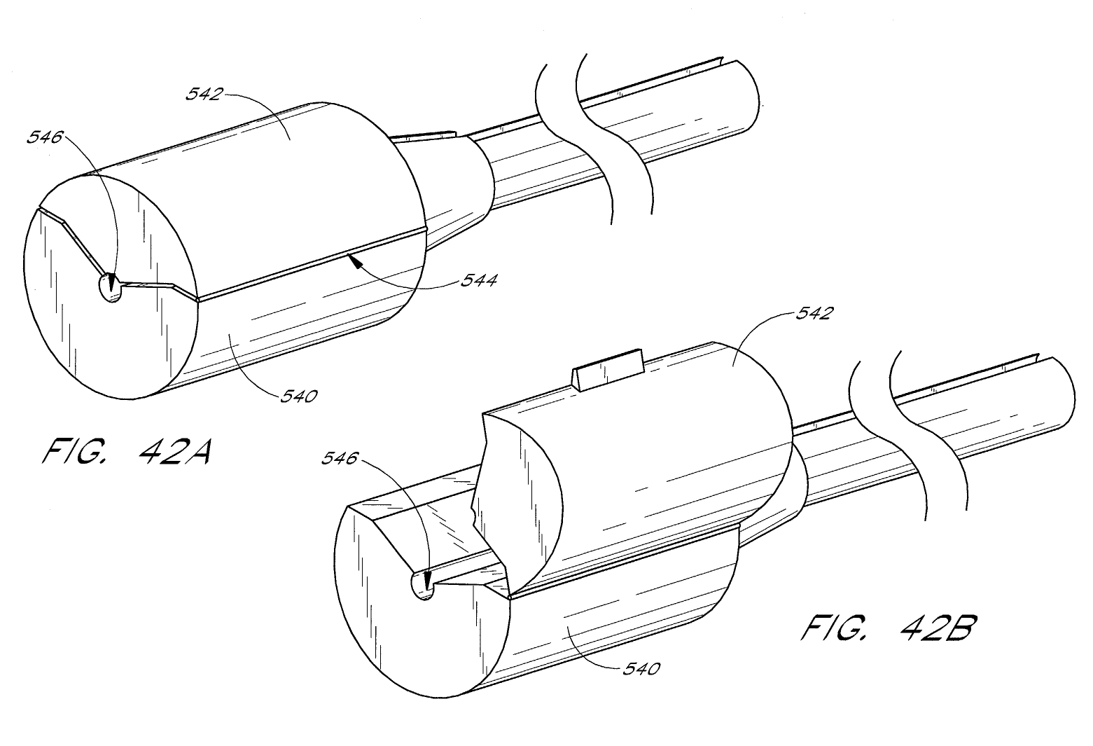

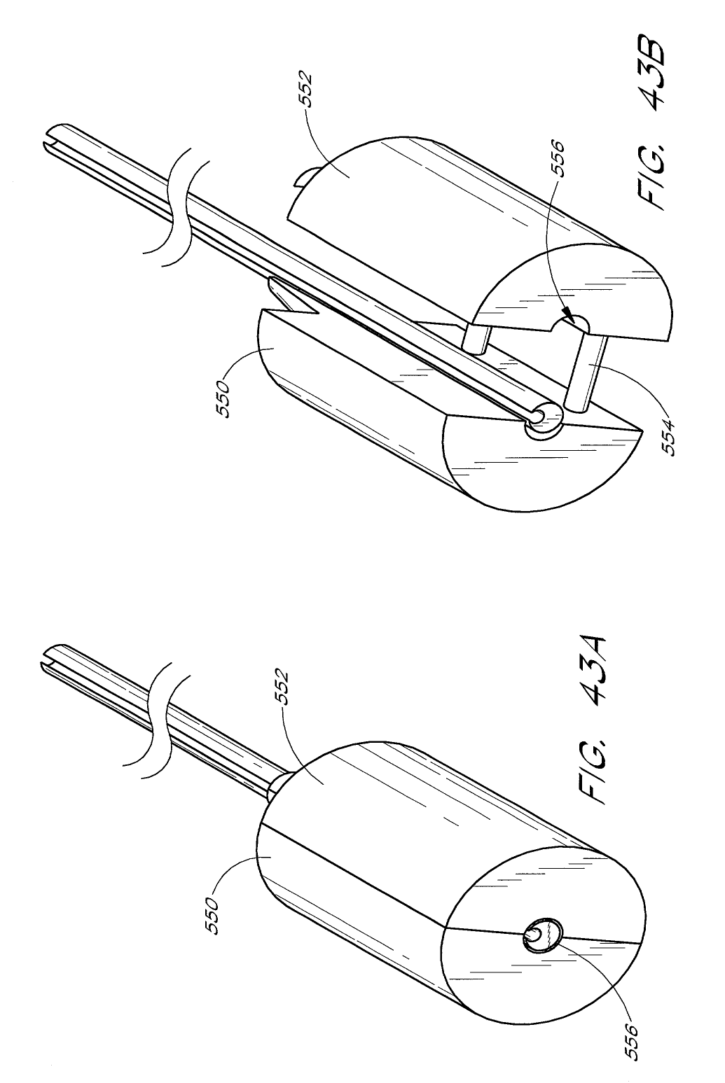

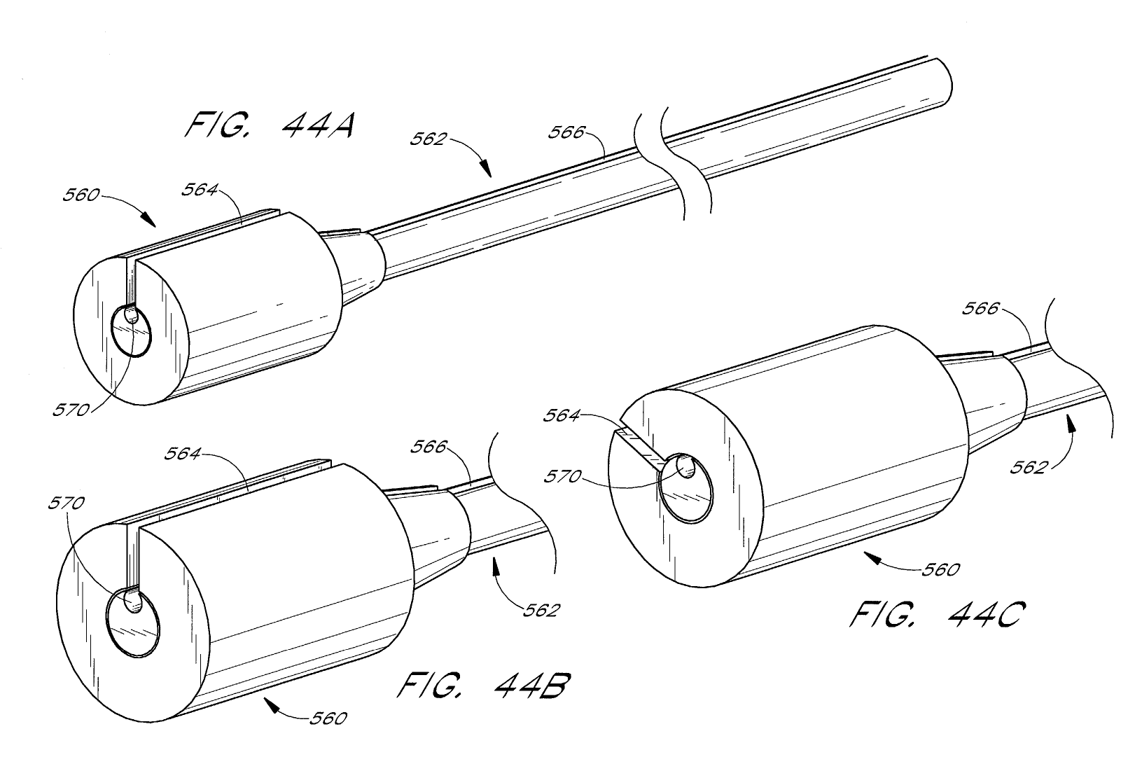

In a variation of the embodiment just described, a split proximal hub is provided at the proximal end of the protective sheath assembly comprising a distal portion 522 formed with a distal slot 524 and a proximal portion 526 formed with a proximal slot 528 as illustrated in FIGS. 37-41. The proximal portion 526 can be rotated relative to the distal portion 522 to move the proximal slot 528 out of alignment with the distal slot 524. A dowel pin 530 is preferably provided to limit the rotation of the hub. FIG. 37A shows the locking hub with the distal slot 524 and proximal slot 528 in alignment. FIG. 37B shows the locking hub with the distal slot 524 and proximal slot 528 out of alignment. FIG. 38A illustrates a cross-sectional view of the locking hub showing the distal slot 524 and proximal slot 528 in alignment. FIG. 38B illustrates a cross-sectional view with the distal slot 524 and proximal slot 528 out of alignment. FIG. 39 is a perspective view of the rotating split-hub with the distal slot 524 and proximal slot 528 out of alignment. FIG. 40 is an exploded view showing the assembly of one preferred embodiment of a rotating hub. The proximal portion 526 of the hub mates with the distal portion 522 of the hub. The dowel pin 530 is inserted through a hole 523 in the distal portion 522 and extends through the distal portion 522 and into a slot 527 in the proximal portion 526. FIG. 41 is an exploded view showing the assembly of a slight variation of the split hub wherein the dowel pin 531 extends through a hole 525 in the proximal portion 532 into a slot 538 in the distal portion 536.

FIGS. 42A and 42B disclose another embodiment of the proximal hub whereby comprising a main portion 540 and a flip tab portion 542 attached by a hinge 544. FIG. 42B shows the proximal hub with the flip tab portion open such that the filter shaft may be inserted into the lumen 546. After the filter shaft has been inserted into the lumen, the hub is closed to prevent the filter shaft from falling out as shown in FIG. 42A.

FIGS. 43A and 43B disclose another embodiment of the proximal hub comprising two separable halves 550 and 552. The two separable halves are connected by a pin 554 (or similar member) and may be separated to insert the filter shaft into the lumen and then pushed back together to lock the filter shaft in the lumen. FIG. 43B illustrates this embodiment of a locking hub with the two separable halves separated such the filter may be inserted into the lumen 556.

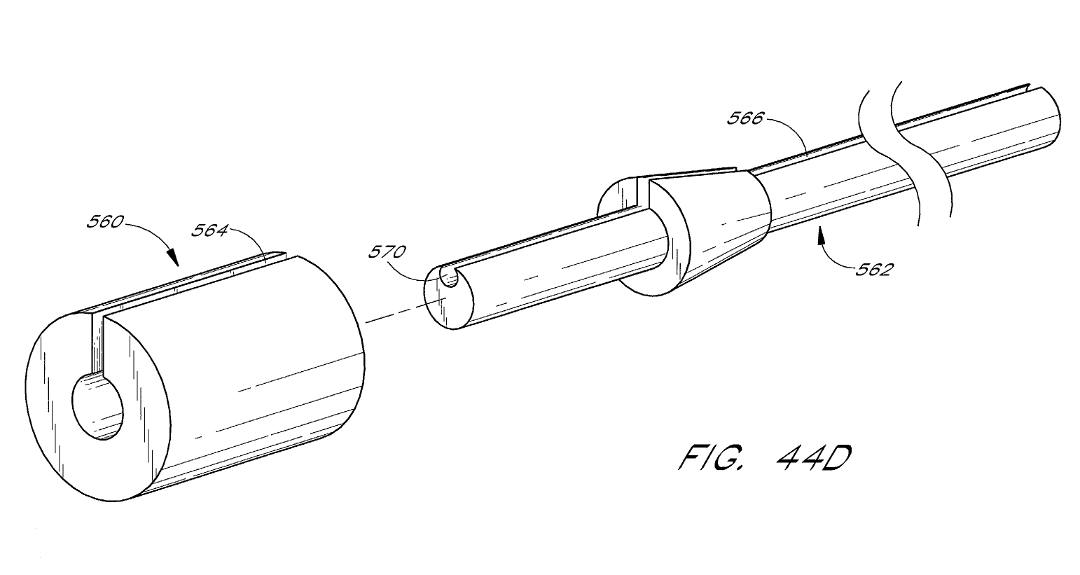

FIGS. 44A-44D disclose yet another embodiment of the proximal hub wherein the proximal hub 560 rotates independently of the elongated body 562 such that the slot 564 in the hub 560 may be moved out of alignment with the slot 566 in the elongated body 562. FIG. 44B illustrates the device with the slot 564 in the hub 560 aligned with the slot 566 in the elongated body 562. In this configuration, the filter shaft can be easily side-loaded into the sheath. With the filter shaft loaded into the lumen 570 of the protective sheath assembly, the hub 560 can be rotated to move the respective slots out of alignment, as shown in FIG. 44C. With the slots out of alignment, the filter shaft cannot be removed from the lumen 570. FIG. 44D is an exploded perspective view of this embodiment showing the hub 560 and elongated body 562 as separate components.

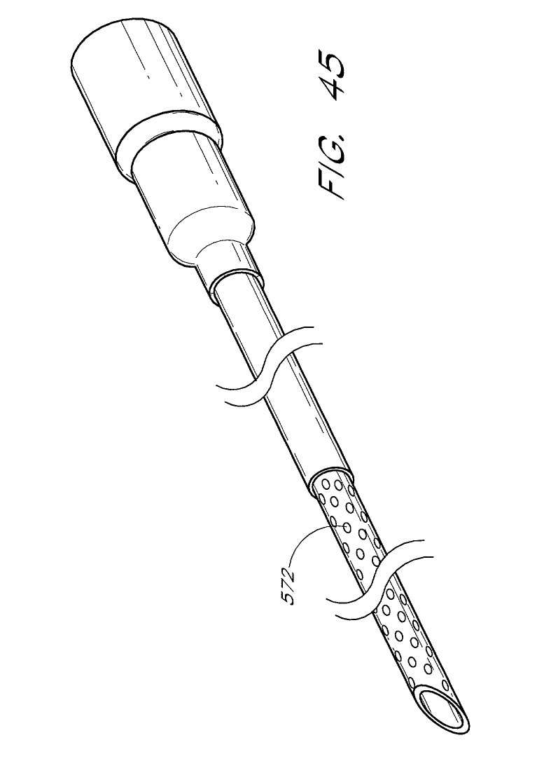

FIG. 45 shows an additional embodiment of a protective sheath wherein the distal portion of the protective sheath is formed with a plurality of holes 572. The holes 572 aid the user in flushing the protective sheath with a fluid to facilitate the removal of air from the lumen in the sheath.

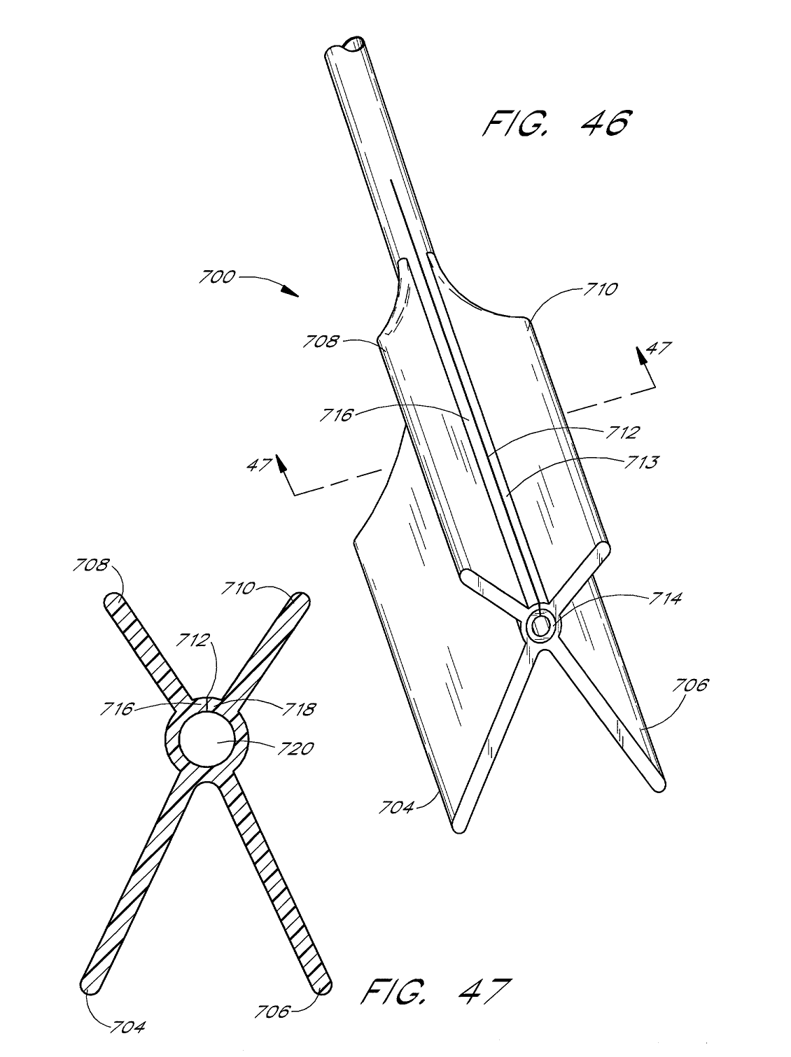

FIGS. 46-49 illustrate yet another embodiment of a protective sheath 700 wherein a longitudinal slot 712 is provided along the proximal end portion of the protective sheath for loading an intravascular device into the lumen 720 of the sheath. To enable the user to easily open and close the longitudinal slot 712, this embodiment is provided with two projections 704, 706 that extend radially from the proximal end portion on the opposite side from the longitudinal slot 712.

When the protective sheath 700 is in a relaxed state, as shown in FIGS. 46-47, the opposing walls 716, 718 of the sheath are in contact along the longitudinal slot 712. In this configuration, the proximal slot 712 is closed and maintains a seal around the interior lumen 720 of the protective sheath. FIG. 47 is a cross-sectional view of the proximal end portion of the protective sheath illustrating the device in the relaxed configuration such that the opposing walls 716, 718 are in contact such that a seal is maintained around the lumen 720.

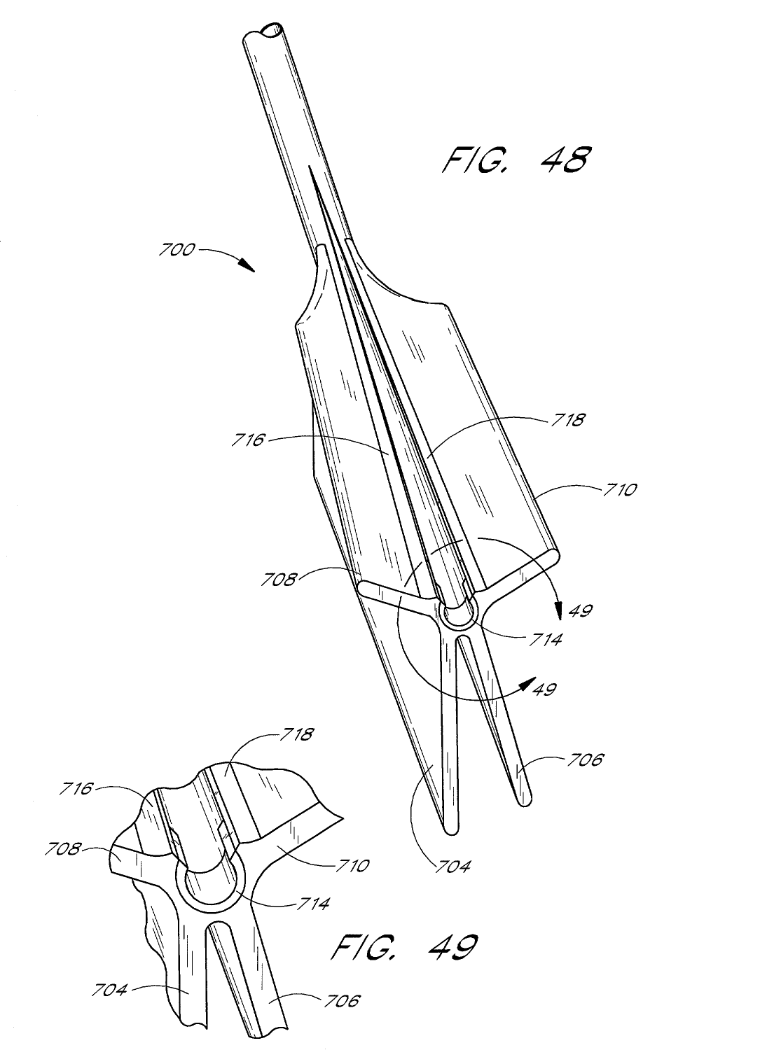

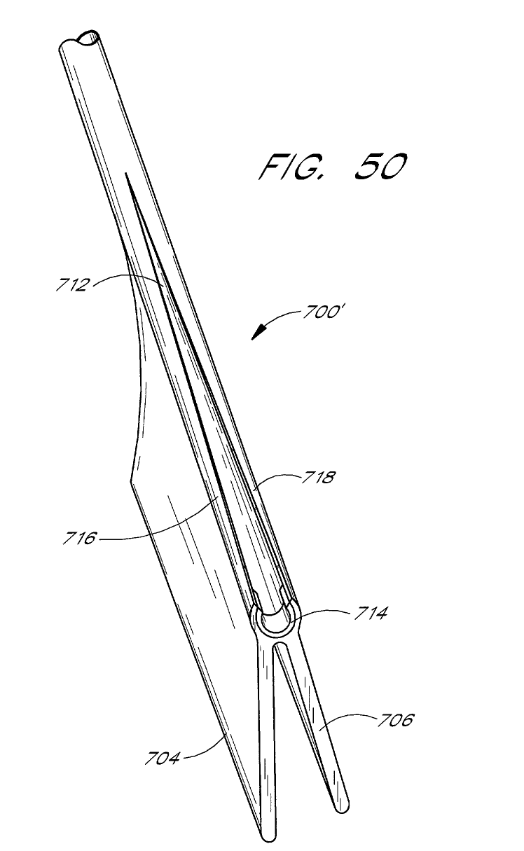

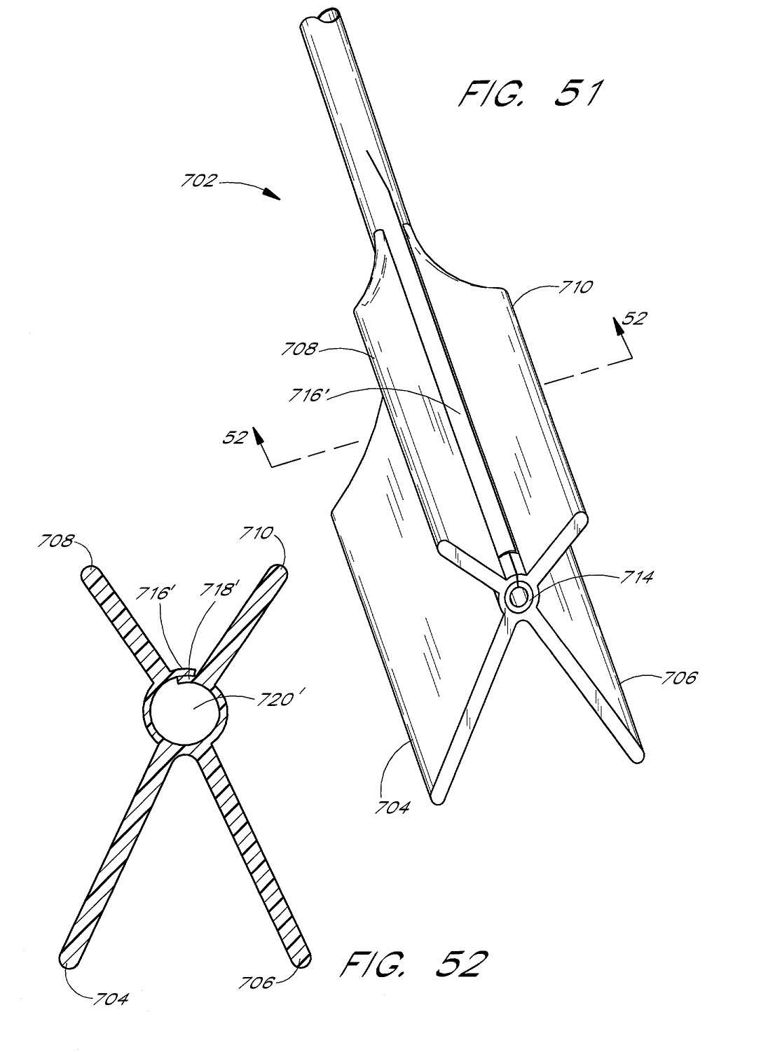

As illustrated in FIG. 48, when the two projections 704, 706 are pinched together, the proximal end portion of the sheath 700 is caused to deform such that the opposing walls 716, 718 of the slot 712 become separated. In this configuration, the interior lumen 720 of the sheath can be advantageously accessed for loading or unloading an intravascular device. FIG. 49 provides an enlarged view of the proximal end of the sheath 700 in the deformed configuration with the interior lumen 720 exposed. When the interior lumen is exposed, the distal portion of a filter device (or other intravascular device) may be quickly and easily inserted through the slot 712 into the lumen 720 of the protective sheath 700. The guide members 708, 710 extending radially along opposite sides of the longitudinal slot 712 are provided to help guide the filter device toward the slot 712 to facilitate the loading of the filter device into the protective sheath.

As best shown in FIGS. 48-49, an annular member 714 is preferably provided within the lumen 720 at the extreme proximal end of the protective sheath 700. The annular member provides a seal at the proximal end of the protective sheath 700 to prevent blood backflow therethrough. The annular member 714 is formed with an orifice 722 having a relatively small diameter as compared with the interior lumen 720 of the sheath 700. As the distal end of the intravascular device is inserted through the slot 712, the wire extending proximally from the device is inserted through the slotted portion of the annular member 714 and into the orifice of the annular member 714.

When the projections 704, 706 are released, the proximal end portion of the protective sheath 702 returns to its relaxed state. In the relaxed state, the diameter of the orifice in the annular member 714 reduces to a size approximately equal to the outer diameter of the wire such that there is minimal clearance therebetween. Therefore, the annular member 714 allows the wire to slide longitudinally through the orifice yet maintains a seal around the wire to minimize blood back flow through the sheath. In the relaxed state, the opposing walls 716, 718 along the longitudinal slot 712 are in contact and thereby provide a seal around the interior lumen of the sheath. The proximal portion of the sheath is preferably made of an elastomeric material that will easily deform to open the slot on the sheath when the projections 704, 706 are squeezed together and will return to its original shape when the projections are released.

FIG. 50 illustrates a variation that is similar to the device just described with reference to FIGS. 46-49; however, this protective sheath 700′ is constructed without guide members.

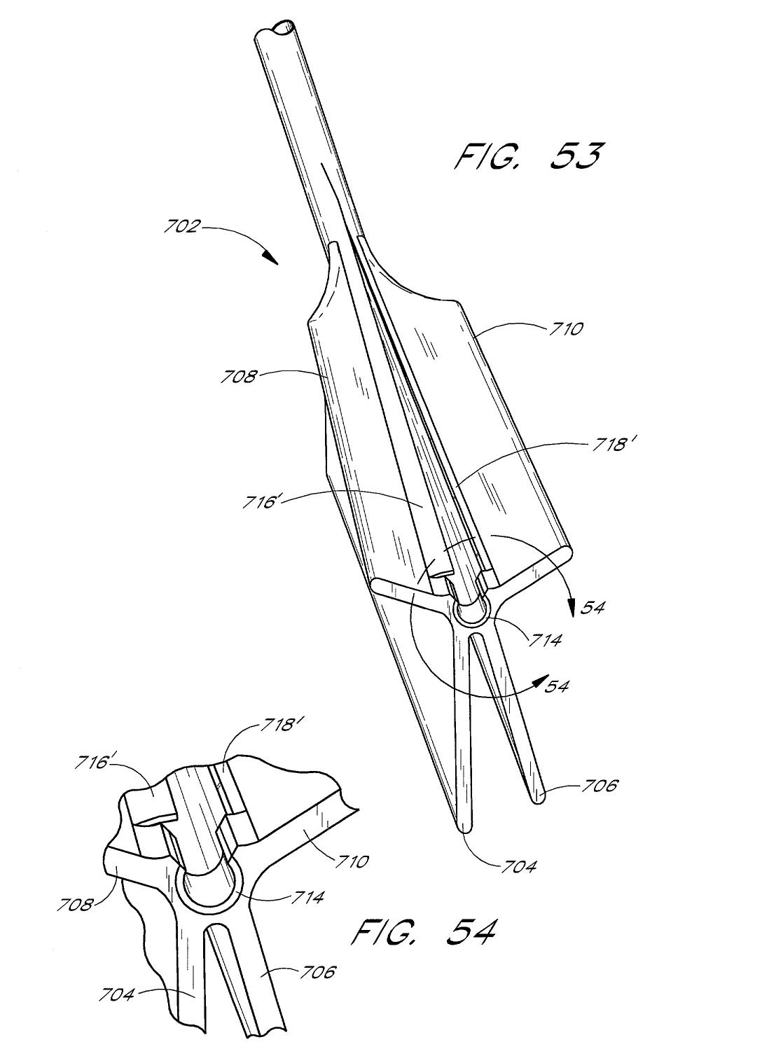

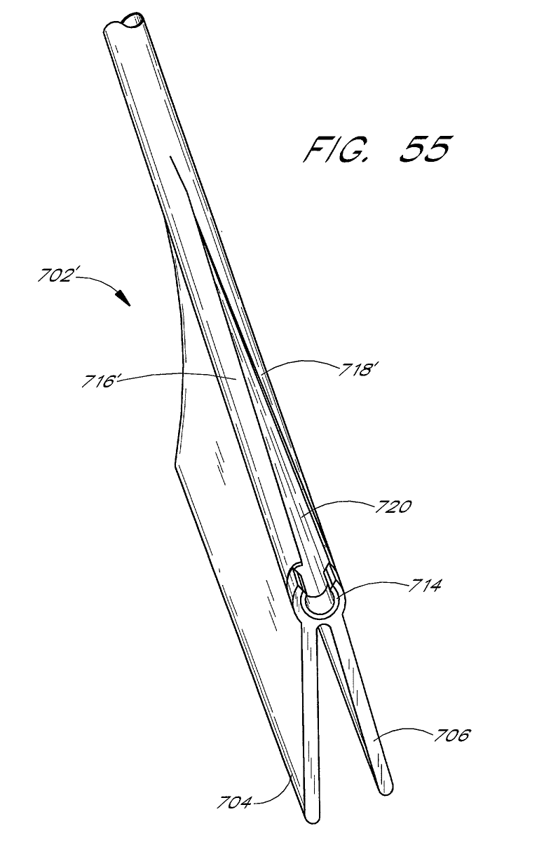

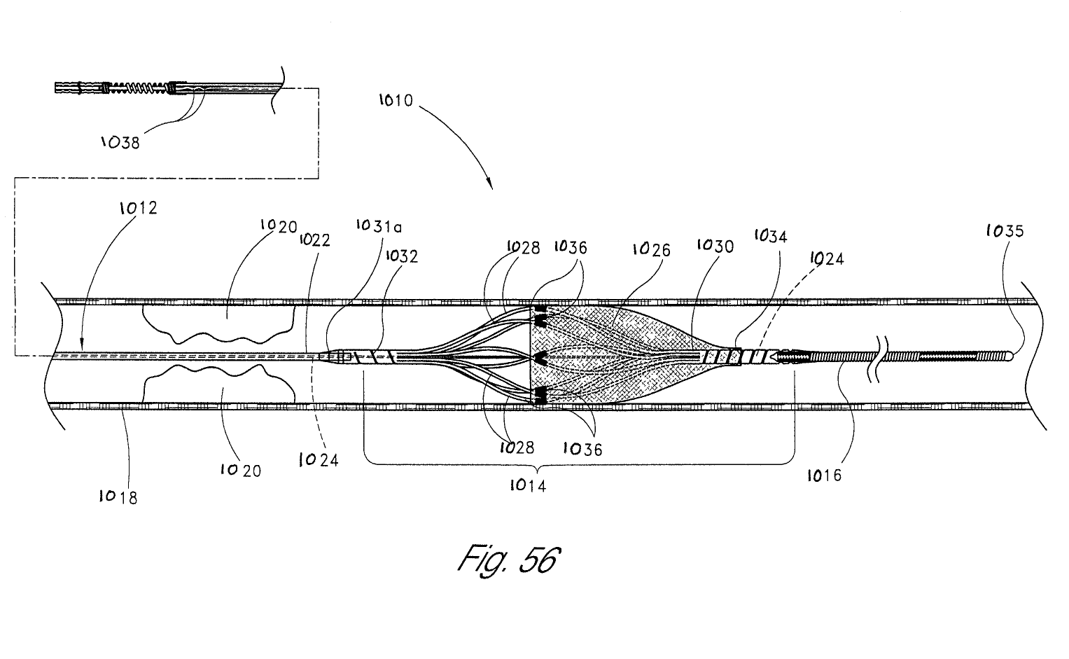

FIG. 51 illustrates another variation wherein the opposing walls 716′, 718′ of the protective sheath 702 are formed to overlap when the sheath is in the relaxed condition. This variation provides an improved sealing mechanism to prevent the escape of blood or other fluid from the lumen 720′ of the protective sheath 702. FIG. 52 is a cross-sectional view of the embodiment of FIG. 51 illustrating how the walls 716′, 718′ of the sheath 702 overlap while the protective sheath is in the relaxed configuration. FIGS. 53-54 illustrate this variation of the protective sheath with the projections 704, 706 squeezed together to expose the lumen 720′ for insertion of the intravascular device.

FIG. 55 illustrates a variation that is similar to the device just described with reference to FIGS. 51-54; however, this protective sheath 702′ is constructed without guide members.

Overview of Occlusion System

FIG. 56 illustrates a preferred embodiment of a filter device 1010 comprising a shaft 1012, a filter subassembly 1014, and a guide tip 1016. An adapter 1118 (see FIGS. 63A-64) may be operably connected to the filter device to expand the filter. Further details of each of these components are described below.

In employing the device 1010, the filter subassembly 1014 is delivered on the shaft 1012 to a location in a blood vessel 1018 distal of an occlusion 1020. Through the use of the adapter 1118, the filter subassembly 1014 is expanded to occlude the vessel distal of the occlusion. Various therapy and other catheters can be delivered and exchanged over the shaft 1012 to perform treatment on the occlusion 1018. Because the filter subassembly 1014 remains expanded distal of the occlusion 1018, any particles broken off by treating the occlusion 1020 are trapped within the filter subassembly. These particles may then be removed by contracting the filter subassembly 1014 so as to contain the particles and withdrawing the device 1010 from the vessel. As an alternative or in addition to this method of particle removal, an aspiration catheter may be delivered over the shaft 1012 and used to aspirate some or all of the particles from the filter subassembly 1014.

Shaft

As shown in FIG. 56, the shaft 1012 comprises an outer shaft member 1022, and a pull wire 1024 which extends through the lumen of the outer shaft member. The outer shaft member 1022 may comprise a hypotube as is known in the art. Moreover, as described in assignee's copending application entitled STRUT DESIGN FOR AN OCCLUSION DEVICE, Ser. No. 09/505,546 filed Feb. 17, 2000, the entirety of which is hereby incorporated by reference, multiple hypotubes may be coaxially disposed over the pull wire 1024. The shaft extends from a proximal end distally to the filter subassembly 1014. The shaft may be constructed to any desired length, however, it is preferable for the shaft to be between about 120 and 300 cm in length.

The size of the outer member of the shaft 1012 is suitable for insertion into the vasculature of a patient through an insertion site in the skin of the patient. It is preferable that the outer shaft member 1022, the pull wire 1024, and any other hypotube members are disposed coaxially such that each member is located within any larger diameter member and surrounds any smaller diameter member.

It is preferable that the largest diameter member of the shaft, for example outer member 1022 in FIG. 56, has an exterior diameter of about 0.009 to 0.035 inches. It is more preferable that the largest diameter member of the shaft has an exterior diameter of about 0.012 to 0.035 inches, more preferably about 0.014 to 0.018 inches, and most preferably about 0.0142 inches. The wall thickness of the largest diameter hollow member of the shaft is preferably about 0.001 to 0.008 inches; i.e. the diameter of the lumen of the largest hollow member of the shaft is preferably from about 0.002 to 0.016 inches less than the outer diameter of the member. Any members located within the largest diameter member are preferably sized so as to fit within the inner lumen of the larger member.

As shown in FIG. 56, the outer member 1022 of the shaft extends distally and is connected at its distal end to the filter subassembly 1014. The pull wire 1024 is the most centrally disposed of the shaft members. The pull wire 1024 is preferably a solid, i.e. non-tubular member around which the outer member 1022 is disposed. The pull wire 1024 preferably extends inside the outer member 1022, through the filter subassembly 1014, and into the guide tip 1016. Alternatively, the pull wire 1024 may have two or more distinct segments, such as a proximal segment which extends to and terminates at the distal end of the strut hypotube 1030 and a distal segment which extends from that point to the distal end of the guide tip 1016.

The shaft members 1022, 1024 are preferably formed from a material which is sufficiently strong to support the shaft 1012 itself as well as the filter subassembly 1014 at the distal end under the tension, compression, and torsion experienced when inserting, operating, and removing the device from the vasculature of a patient. The material is preferably also sufficiently flexible and elastic that it does not develop permanent deformation while being threaded through the curved path necessary to reach the treatment site from the insertion point. In a preferred embodiment, the shaft 1012 has a friction-reducing outer coating of TEFLON®.

In order to satisfy these requirements, it is preferable to use a metallic tube or wire to form the shaft members 1022, 1024, although a braided or non-braided polymer tube may also provide the desired characteristics. More preferably, a superelastic memory alloy such as straight-annealed nitinol is used for the outer shaft member 1022; tempered stainless steel is one preferred material for the pull wire 1024. Other suitable alloys for the shaft members include nitinol-stainless steel alloys, or nitinol alloyed with vanadium, cobalt, chromium, niobium, palladium, or copper in varying amounts. Additional details regarding materials used for the shaft members are disclosed in U.S. Pat. No. 6,068,623, the entirety of which is hereby incorporated by reference.

Filter Subassembly

Still referring to FIG. 56, the filter subassembly 1014 extends from the distal end of the shaft 1012. The filter subassembly 1014 preferably comprises an expandable member which is either integrally formed or separately attached (as shown in FIG. 56) to the distal end of the shaft 1012. The expandable member preferably includes an occlusive member or membrane 1026 and provides support for this occlusive member.

As used herein, “occlusion” or “sealing”, and the like, refer to blockage of fluid flow in a vascular segment, either completely or partially. In some cases, a complete blockage of the blood vessel may not be achievable or even desirable, for instance, when blood flow must be maintained continuously to the region downstream of the occlusive device. In these cases, perfusive flow through the occluded region is desirable and a partial blockage is used. For example, a partial blockage may be produced using an occlusive member whose cross-sectional dimension does not span the entire blood vessel. Alternatively, a partial blockage may be produced using an occlusive member whose cross-sectional dimension does substantially span the entire blood vessel, but which contains openings or other means for flow to move through the occlusive member perfusively. In other cases, a partial blockage may not be achievable or desirable, and an occlusive member which substantially spans the cross section of the blood vessel without allowing perfusion is used. Each of these described structures makes use of “occlusion,” as defined herein.

In the embodiment shown in FIG. 56, the expandable member comprises struts 1028 which are formed in a strut hypotube 1030. The strut hypotube 1030 extends from the distal end of the outer shaft member 1022 to the proximal end of the guide tip 1016. At its proximal end the strut hypotube 1030 is soldered, crimped, and/or bonded, or otherwise affixed to the distal end of the outer shaft member 1022. In a preferred embodiment, a proximal taper 1031a, preferably formed from a flexible UV-cured adhesive, facilitates the connection of the strut hypotube 1030 to the shaft 1012. At its distal end the strut hypotube 1030 is crimped over a solder junction between the pull wire 1024 and the proximal end of the guide tip 1016. A distal taper 1031b, also preferably formed from a flexible UV-cured adhesive, may be employed as well in attaching the strut hypotube 1030 to the guide tip 1016. With the strut hypotube, pull wire and guide tip joined in this manner, a proximal movement of the pull wire with respect to the outer shaft member 1022 causes a corresponding proximal movement of the distal end of the strut hypotube, thus compressing the strut hypotube and urging the struts toward the expanded position.

The strut hypotube 1030 is preferably formed from nitinol, but may alternatively be formed from nitinol-stainless steel alloys, or nitinol alloyed with vanadium, cobalt, chromium, niobium, palladium, or copper in varying amounts. The strut hypotube preferably has an outside diameter of about 0.0213 inches and an inside diameter of about 0.0144 inches.

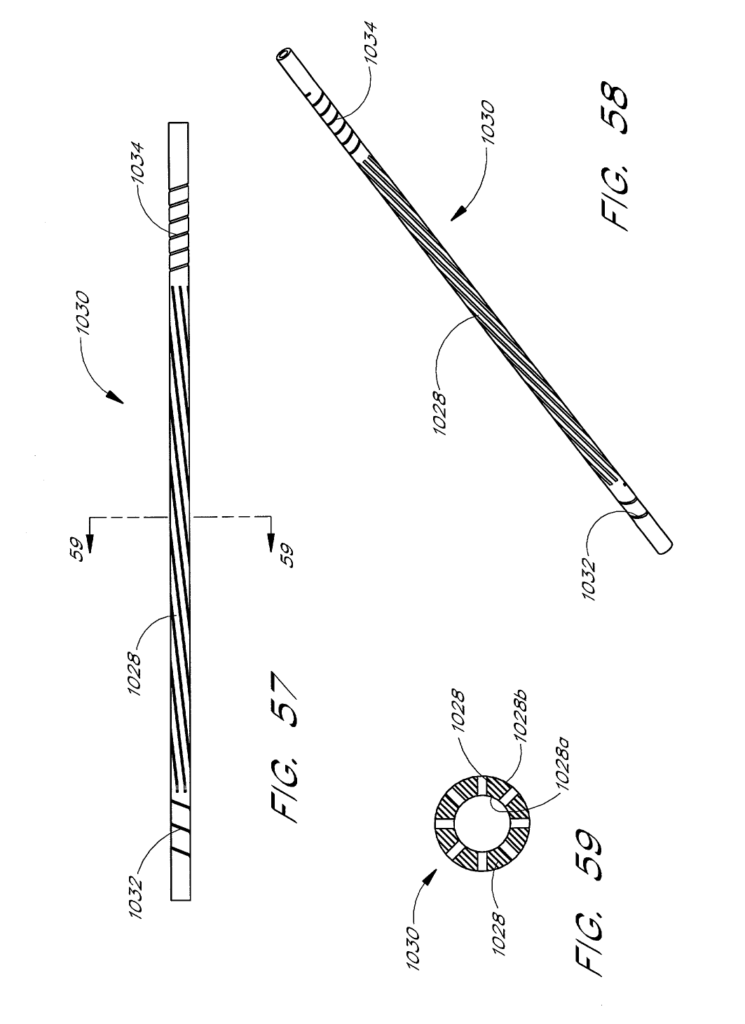

As best seen in FIGS. 57 and 58, the individual struts 1028 are preferably cut from, and thus integral to, the strut hypotube 1030. The struts 1028 may advantageously be formed by subjecting the strut hypotube 1030 to a laser-cutting process. Although the number of struts 1028 may vary, there are preferably between 4 and 10 (most preferably 8) struts. The struts 1028 should be equally radially spaced about the longitudinal centerline of the strut hypotube 1030.

It is preferred that the struts 1028 have a helical configuration, with each strut making approximately 1.0 revolution, at a substantially constant pitch, about the longitudinal centerline of the strut hypotube 1030 as it extends from its proximal to its distal end. Alternative preferred embodiments have straight slits which provide for non-spiral struts when deployed into the expanded configuration. The preferred helical configuration improves the apposition of the struts against the vessel wall when the filter subassembly is in the expanded configuration. The struts 1028 may advantageously have a constant clockwise pitch of about 0.650 inches and therefore the portion of the hypotube into which the struts are cut is about 0.650 inches in length. It is contemplated that the filter subassembly should reach a preferred maximum diameter of about 7.5 mm when expanded. As used herein, “strut” refers to any mechanical structure which extends from another structure or which is used to support a membrane or other structure of the occlusion device. Specifically, as discussed herein, the struts of the occlusion device are those portions of the device which extend from the shaft in order to adjust the profile of the device as discussed below, and which may be used to support the membrane.

FIG. 59 depicts a cross-section of the strut hypotube 1030, taken along the line A-A as shown in FIG. 57. The preferred laser-cutting process creates a gap of about 0.0018 inches in width between each pair of struts 1028. Each strut 1028 thus has a preferred cross-section that comprises an angular section of an annulus, with a smaller-radius inner surface 1028a and a broader, larger-radius outer surface 1028b. By virtue of their increase in size near the outer surface 1028b, the struts 1028 are stronger than a comparable set of struts that have a simple rectangular cross-section and are sized to fit within the same inner diameter-outer diameter “envelope.”

With further reference to FIGS. 57 and 58, the strut hypotube 1030 may preferably incorporate a proximal cut 1032 and/or a distal cut 1034, to improve the flexibility of the hypotube. Each of the cuts 1032, 1034 is helical, with the proximal cut 1032 having a preferred substantially constant pitch of about 0.030 inches and the distal cut 1034 having a preferred substantially constant pitch of about 0.020 inches. The proximal cut 1032 and distal cut 1034 preferably extend along about 0.075 inches and 0.125 inches, respectively, of the hypotube 1030 (as measured along its longitudinal axis), and each has a preferred cut width of about 0.0018 inches. Preferably, an uncut “gap” of about 0.015 inches exists on the strut hypotube 1030 between the proximal cut 1032 and the proximal end of the struts 1028, and between the distal cut 1034 and the distal end of the struts. As shown in FIG. 56, when the strut hypotube 1030 is attached to the shaft 1012 and the guide tip 1016, it is advantageous that no part of the cuts 1032, 1034 overlie any portion of the shaft or guide tip, so as not to impede the flexibility enhancement that is provided to the strut hypotube by the cuts.

In a preferred embodiment, one or more marker bands 1036 (see FIG. 56) are attached to a corresponding number of the struts, and are advantageously located at or near the midpoint of each strut, so as to align the marker bands with the widest portion of the filter subassembly 1014 when it is in the expanded configuration. The marker bands may thus be aligned in a plane extending substantially orthogonal to the longitudinal axis of the shaft 1012. Alternatively, the marker bands 1036 may be staggered, i.e. attached in varying locations along the length of the struts 1028, in order to reduce the profile of the filter subassembly when it is in the collapsed configuration. The marker bands are advantageously configured to wrap around only three sides of each strut, leaving the outer surface 1028b (see FIG. 59) exposed, in order to reduce the profile of the filter subassembly when it is in the expanded configuration. A proximal marker band (not shown) may be incorporated in a location proximal of the struts 1028 to mark a point on the device beyond which a catheter positioned on the shaft 1012 should not be advanced, thus preventing inadvertent collapse of, or damage to, the struts 1028. A preferred location for the proximal marker band is at the junction of the shaft 1012 and the strut hypotube 1030, underlying the proximal taper 1031a. Additional details not necessary to mention here may be found in U.S. Pat. No. 6,228,072, the entirety of which is hereby incorporated by reference.

The marker bands 1036 are formed from a material having increased radiopacity in comparison to the rest of the filter subassembly, such as platinum, gold, or alloys thereof. In a preferred embodiment, the marker bands comprise an alloy of 80% platinum and 20% iridium. Additional details not necessary to mention here may be found in assignee's copending application entitled VASCULAR FILTERS WITH RADIOPAQUE MARKINGS, U.S. Ser. No. 09/747,175, filed on Dec. 22, 2000 [Attorney Docket PERCUS.087CP1], the entirety of which is hereby incorporated by reference.

As shown in FIG. 56, the pull wire 1024 extends past the distal end of the outer shaft member 1022, beyond the strut hypotube 1030, and terminates in a solder joint 1035 at the distal end of the distal tip 1016. The tip 1016 distal to the struts 1028 preferably includes a radiopaque coil material, most preferably platinum, extending between the distal end of the strut hypotube and the solder joint 1035 to aid the practitioner in positioning the expandable member 1014 within the vessel 1018.

The membrane 1026 is preferably attached at its proximal end to the struts 1028, at or proximal of the struts' widest extent when in the expanded configuration. It is also preferred that the membrane 1026 is attached at its distal end to the strut hypotube at or adjacent the distal cut 1034. Between these proximal and distal points of attachment, the membrane tapers gradually to a smaller diameter but preferably tapers less sharply than the distal portion of the struts 1028, so as to remain free from the struts, in a relatively loose or “baggy” state. When the expandable member is deployed, this “baggy” membrane creates a rather deep pocket for catching emboli as blood flows through the membrane 1026, and for containing the emboli when the expandable member is collapsed and withdrawn from the vessel 1018.

Alternatively, the membrane 1026 may be attached to the struts 1028 at one or more points, or in a continuous attachment, between the proximal and distal ends of the membrane. Many other arrangements are possible for the structure and attachment of the membrane 1026. Reference may be made to assignee's copending patent applications U.S. Ser. No. 09/505,554, entitled MEMBRANES FOR OCCLUSION DEVICE AND METHODS AND APPARATUS FOR REDUCING CLOGGING, filed Feb. 17, 2000, and U.S. Ser. No. 09/788,885, entitled MEMBRANES FOR OCCLUSION DEVICE AND METHODS AND APPARATUS FOR REDUCING CLOGGING, filed on Feb. 20, 2001, the entirety of each of which is hereby incorporated by reference. As used herein, “filter” and like terms mean any system which is capable of separating something out of a portion of the blood flow within the vascular segment, whether or not there is perfusion through the “filter”. “Filtering” and similar terms refer to the act of separating anything out of a portion of the blood flow.

The membrane 1026 has a number of pores (not shown) of a suitable size to trap emboli while permitting blood to flow through, and are thus about 20-100 microns in size. Suitable nonelastomeric materials for the membrane 1026 include polyurethane, polyethylene, polyethylene terephthalate (PET), expanded polytetrafluoroethylene (PTFE), and polyether-based polyamides sold under the trade name PEBAX by Elf Atochem. One suitable elastomeric material is a block copolymer of styrene-ethylenebutylene-styrene (SEBS), available under the trade name C-FLEX, sold by Consolidated Polymer Technologies. The membrane may also be made from latex or silicone. The membrane may alternatively comprise a polymer mesh of polyurethane, nylon, polyester, or polyethylene, with pores approximately 30-50 microns in diameter. Yet another alternative is a braid of polyester or nitinol. To prevent formation of blood clots on the occlusive member, it may be coated with heparin or other known antithrombogenic agents such as hirudin or pirudin.

Most preferably, the membrane 1026 is formed from polyurethane and has pores of about 100 microns in size, or a combination of pore sizes within the ranges detailed above. The pores are preferably spaced apart on the membrane with about 0.010 inches between the centers of adjacent pores. It is also preferred that the proximal portion of the membrane lack pores, to facilitate bonding the membrane to the struts 1028 over the marker bands 1036. Likewise, the distal portion of the membrane may also be nonporous, providing easier attachment to the strut hypotube 1030.

Further details not necessary to repeat here are disclosed in assignee's copending application entitled OCCLUSION OF A VESSEL, Ser. No. 09/374,741, filed Aug. 13, 1999, the entirety of which is hereby incorporated by reference.

Pull Wire

The outer shaft member 1022 surrounds the pull wire 1024 and is connected to the strut hypotube 1030 at its proximal end (see FIG. 56). The pull wire 1024 is advantageously attached to distal end of the strut hypotube 1030, so that when the pull wire 1024 is retracted relative to the outer shaft member 1022, the struts 1028 are urged to expand in a radial direction. The relative position of the outer shaft member 1022 and the pull wire 1024 is varied until the vessel 1018 is occluded. The struts 1028 bow outwards toward the wall of the vessel 1018, so that the filter subassembly 1014 seals the vessel 1018 (i.e., in its deployed position, the expandable member prevents emboli from moving downstream). The radial expansion of the struts 1028 may also be facilitated by advantageously imparting an initial curvature to the struts 1028 through heat setting. The pull wire 1024 may advantageously extend within the distal guide tip 1016 beyond the distal end of the strut hypotube 1030 and terminate in the solder joint 1035 at the distal end of the guide tip.

After the filter subassembly 1014 is deployed, the struts 1028 tend towards their collapsed, undeployed position in the absence of a restraining force (unless the filter subassembly 1014 is self-expanding, in which case the filter subassembly has a tendency to remain in the deployed position). To prevent the struts from returning to their undeployed position, the pull wire 1024 has one or more bends 1038 formed therein for contacting the inner wall of the outer shaft member 1022, thereby providing frictional forces which keep the filter subassembly 1014 in its expanded, deployed position, as shown in FIG. 56. Specifically, the frictional force between the pull wire 1024 and the outer shaft member 1022 is sufficient to offset or compensate for the spring force provided by the struts 1028 and/or the membrane 1026, which would otherwise urge the struts towards their relaxed position. Whereas 0.5-1 pound of pulling force may be required to expand the struts 1028, the friction between the pull wire 1024 and the filter subassembly 1014 may be sufficient to restrain up to 3 pounds of pulling force. Thus, the bends 1038 of the pull wire 1024 engage the outer shaft member 1022 to form a compact device for restraining the pull wire from unwanted longitudinal motion. The bends 1038 of the pull wire 1024 may be formed, for example, by coining or by forming a spring in the pull wire. The bends 1038 thus act as a locking member which inhibits movement of the pull wire 1024, and the pull wire 1024 and the outer shaft member 1022 are frictionally secured together.

The pull wire features of the embodiment of FIG. 56 can also be used if the filter subassembly 1014 is shape set so that it tends toward an expanded, deployed position in the absence of any applied forces, i.e. if the expandable member is self-deploying. In the case where an embodiment such as that shown in FIG. 56 is constructed using a self-deploying filter subassembly 1014, the pull wire 1024 effectively acts as a push-wire which holds the filter subassembly in the collapsed configuration. This push-wire is held in place by the frictional engagement between the bends 1038 of the pull wire and the outer shaft member 1022.

When using such a device as shown in FIG. 56 with an expandable member which is self-deploying, the filter subassembly 1014 is inserted into the vessel 1018 of the patient in its low profile position, with frictional forces between the pull wire 1024 and the outer shaft member 1022 holding the pull wire 1024 in the distal direction, which prevents the filter subassembly from expanding. The filter subassembly 1014 is then deployed by urging the pull wire 1024 in the proximal, axial direction (retracting the pull wire) with sufficient force to overcome the frictional forces between the pull wire 1024 and the outer shaft member 1022, thereby moving the locking member 1038 out of its locked position. In effect, by moving the pull wire proximally in this way, the “pushing” effect of the pull wire is eliminated, and the expandable member will deploy into the expanded configuration.

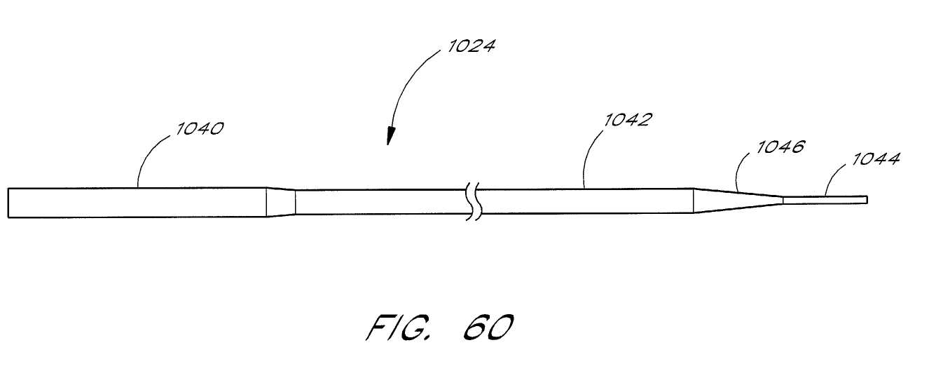

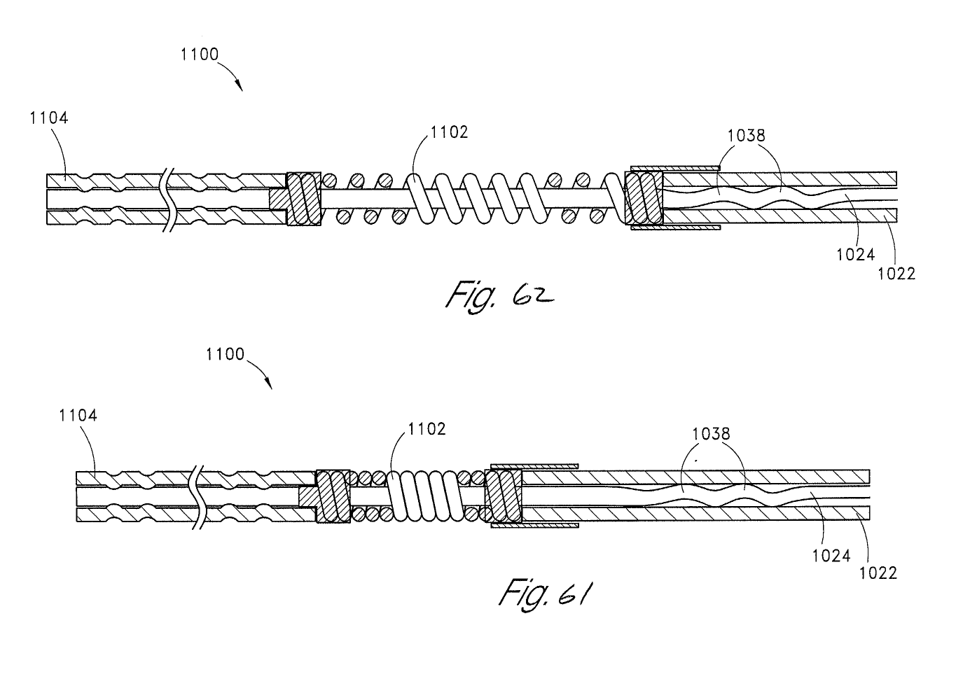

FIG. 60 shows one preferred embodiment of the pull wire 1024. A preferred pull wire 1024 comprises a tempered stainless-steel wire with an anti-friction coating of TEFLON®. This pull wire 1024 has a tapered configuration, with a proximal section 1040 having a diameter of about 0.0086 inches; advantageously, this larger-diameter proximal section of the pull wire includes the bends 1038 described above. Distal of this section the pull wire tapers to a medial section 1042 having a diameter of about 0.0070 inches. The pull wire shown has a diameter of about 0.0025 inches at its most distal section 1044; this diameter advantageously prevails over the most distal 3 cm of the pull wire. A tapered transition 1046 of about 3 cm in length is interposed between the medial section and the distal section. The pull wire of FIG. 60 has an overall length of about 212.0 cm; the proximal section (having the diameter of about 0.0086 inches) is about 17.0 cm in length. The medial section is thus about 189.0 cm in length.

Pull Wire Kink Protection

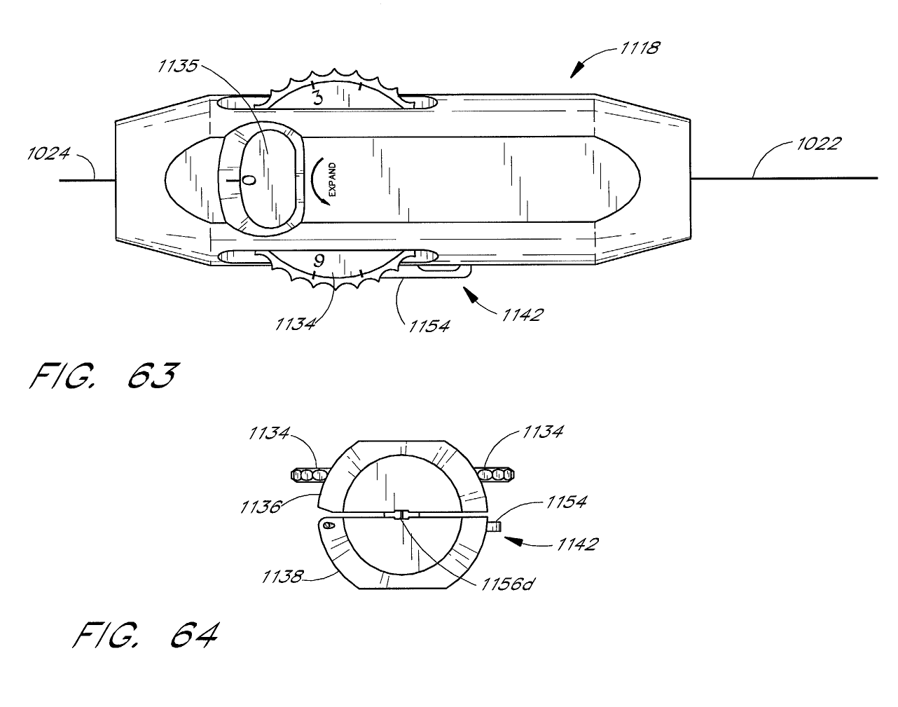

FIGS. 61 and 62 depict a kink protection system 1100 that may preferably be used to prevent the proximal portion of the pull wire 1024 from kinking when it is pushed distally against the frictional resistance of the bends 1038 and, where the filter subassembly 1014 is of the self-expanding type, against the spring force of the struts 1028. The system 1100 comprises a pre-expanded coil 1102 and a proximal hypotube 1104. The coil 1102 is connected to the proximal tip of the outer shaft member 1022 by soldering or other conventional methods and surrounds that portion of the pull wire 1024 which is immediately proximal of the outer shaft member. The proximal hypotube 1104 is crimped to the pull. wire 1024 and is attached to the proximal end of the coil 1102 by soldering or other conventional methods.

FIG. 61 shows the system 1100 when the filter subassembly is in its contracted configuration, and the coil 1102 is compressed. FIG. 62 shows the system 1100 when the filter subassembly is in the expanded configuration. The pull wire 1024 has been pulled proximally from the outer shaft member 1022 and the coil 1102 is in its relaxed state. When the pull wire 1024 is pushed back distally into the outer shaft member 1022 (see FIG. 61), the coil 1102 augments the column strength of the pull wire 1024 by presenting a coaxial, larger-diameter column for absorbing the compressive force that is applied to the coil-pull wire assembly. Off-axis loads are thus less likely to bend or kink the pull wire 1024 as it is pushed into the outer shaft member 1022. Additional details not necessary to mention here may be found in assignee's copending application entitled METHOD AND APPARATUS FOR PROTECTING THE PROXIMAL END OF AN OCCLUSIVE DEVICE FOR EMBOLI CONTAINMENT, Ser. No. ______ [Attorney Docket PERCUS.141A], filed on the same day as the present application, the entirety of which is hereby incorporated by reference.

Adapter

The pull wire 1024, shown in FIG. 56, is manipulated through the use of an adapter or manifold 1118 (see FIGS. 63-66). The adapter enables the technician to control the relative positioning of the pull wire 1024 and the outer shaft member 1022 in a simple manner. Although FIGS. 63-66 illustrate the adapter as manipulating the pull wire 1024, it will be appreciated that in embodiments wherein a proximal hypotube is provided over the pull wire 1024, the adapter manipulates this proximal hypotube.

After delivery of the device to the desired location within the vasculature of the patient, the adapter 1118 is attached and the pull wire 1024 is manipulated through the use of the adapter 1118 so as to deploy the filter subassembly 1014 of the device. At this point, the adapter may be removed from the device so that therapy may be performed.

One type of adapter 1118 used in accordance with preferred embodiments of the filter device is shown in FIGS. 63-65. Without regard to whether the expandable member is of the shape set variety (self-expanding) or is undeployed when relaxed, the degree to which the expandable member is deployed can be monitored by noting the longitudinal position of the pull wire 1024. This allows the user to carefully control the extent to which the expandable member is deployed. A thumb wheel 1134 is used to control the position of the pull wire 1024 relative to the outer shaft member 1022, thereby controlling the extent to which the filter subassembly 1014 of FIG. 56 is expanded. As illustrated by the view of FIGS. 63-65, the adapter 1118 includes two halves 1136, 1138 preferably formed of medical grade polycarbonate or the like.

The two halves 1136, 1138 are attached by at least one hinge 1140, so that the halves are joined in a clam shell manner. A latch 1142 secures the two halves 1136, 1138 while the adapter 1118 is in use. The latch includes a pair of flexible, resilient latching members 1144, 1146 which are mounted within the half 1138. A space 1148 between the two latching members 1144, 1146 receives a locking pin 1150 which has a beveled head 1152. The head 1152 passes through the space 1168 and past the latching members 1144, 1146. The latching members 1144, 1146 prevent the locking pin 1180 from backing out past the latching members which would open up the adapter 1118. To open the halves 1136, 1138, the latching members 1144, 1146 are separated slightly by depressing a flexure member 1154, which pries apart the latching members slightly, thereby freeing the locking pin 1150.

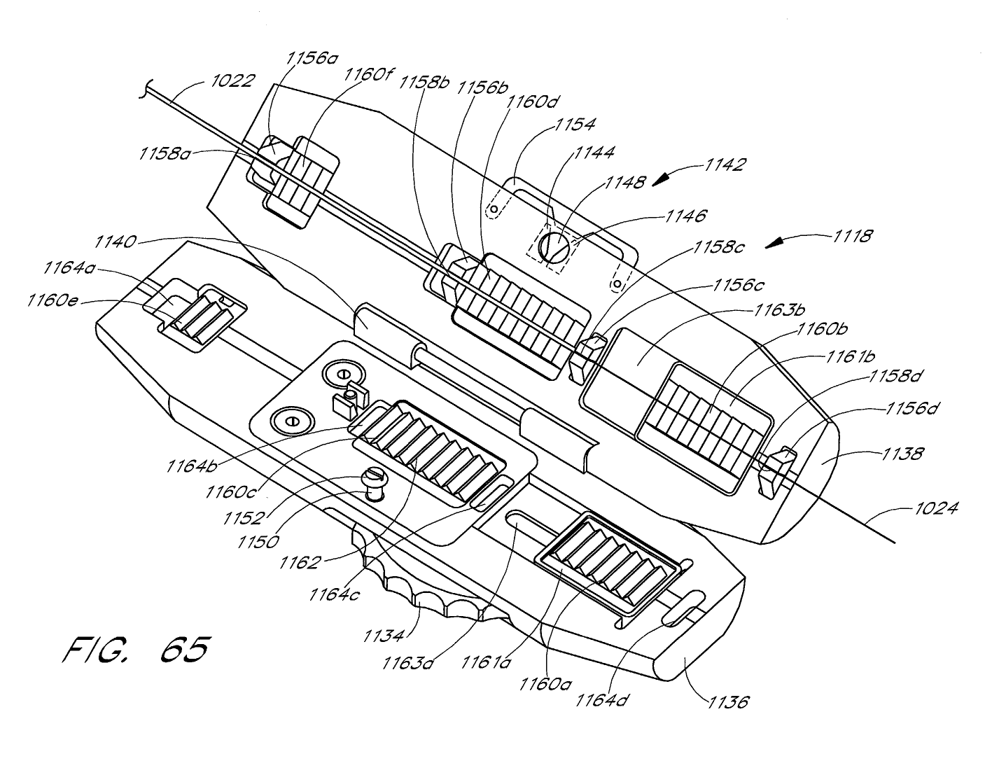

The outer shaft member 1022 may be held in place by a groove (not shown) having a width selected to accept the outer shaft member 1022. Alternatively, as shown in FIG. 65, the outer shaft member 1022 and the pull wire 1024 may be held by clips 1156a, 1156b, 1156c, 1156d having respective slots 1158a, 1158b, 1158c, 1158d therein for receiving the outer shaft member and the pull wire. In particular, the outer shaft member 1022 and the pull wire 1024 may advantageously be configured so that the outer shaft member rests within clips 1156a, 1156b, 1156c, with the pull wire extending between the clip 11 56c and the clip 11 56d and extending proximal to the clip 1156d. With this arrangement, and when the adapter 1118 is in the closed position, the pull wire 1024 may be engaged and moved by a first pair of contact members such as oppositely facing pads 1160a, 1160b, while the outer shaft member 1022 is held stationary by one or more other pairs of oppositely facing pads 1160c, 1160d and 1160e, 1160f. Alternatively, the device may be designed so that the outer shaft member 1022 is moved while the pull wire 1024 remains stationary. The pads 1160a-f may advantageously include a plurality of ridges 1162 for securely contacting the pull wire 1024. The clips 1156a, 1156b, 1156c, 1156d fit within respective cavities 1164a, 1164b, 1164c, 1164d in the adapter half 1136 when the two halves 1136, 1138 are closed.

To aid the user in properly aligning the outer shaft member 1022 and the pull wire 1024 within the adapter 1118, a mark may be placed on the outer shaft member 1022. For example, an alignment mark on the outer shaft member 1022 may indicate that point on the outer shaft member 1022 which must be placed within the slot 1158a so that the outer shaft member extends within the adapter 1118 up to but not proximally beyond the clip 1156c, with the pull wire 1024 being exposed proximal to the clip 1156c. This configuration permits the pads 1160a, 1160b to retract (or advance) the pull wire 1024 into (or out of) the vessel while the outer shaft member 1022 is held securely within the pads 1160c, 1160d and 1160e, 1160f.

When the pull wire 1024 is not being advanced or retracted through the outer shaft member 1022 by the pads 1160a, 1160b, relative movement of the pull wire and the outer shaft member is advantageously prevented by frictional contact between the bends 1038 of the pull wire 1024 and an inner surface of the outer shaft member 1022 (see FIG. 56). This permits the introduction of a therapy catheter (not shown) such as an angioplasty or stent catheter, or the exchange of a plurality of catheters, after the adapter 1118 is decoupled and removed from the outer shaft member 1022 and the pull wire 1024. For example, once the filter subassembly 1014 is deployed, an angioplasty or stent catheter may be introduced over the outer shaft member 1022 and the pull wire 1024. After therapy is performed, an aspiration (and/or irrigation catheter) may be introduced over the outer shaft member 1022/pull wire 1024 to aspirate (and/or irrigate) away emboli entrained in the filter subassembly 1014 which were produced as a result of the therapy procedure. The adapter 1118 may then be recoupled to the outer shaft member 1022 and the pull wire 1024, followed by deactivation (retraction) of the filter subassembly. The filter subassembly 1014, the pull wire 1024, and the outer shaft member 1022 may then be removed from the vessel.

When the adapter 1118 is in the closed position, the pads 1160c, 1160d, 1160e, 1160f surround and contact the outer shaft member 1022 to prevent its motion. The pads 1160a, 1160b, on the other hand, are mounted in respective holders 1161a, 1161b which are slidable within respective recessed portions 1163a, 1163b of the adapter 1118, so that when the pads 1160a, 1160b, surround and contact the pull wire 1024, the pull wire may be retracted or advanced. Specifically, the holder 1161a (housing the pad 1160a) is mechanically coupled to and controlled by the wheel 1134, as discussed in more detail below. When the adapter 1118 is closed, the pads 1160a and 1160b are compressed together and squeeze the pull wire 1024 between them. As the user rotates the wheel 1134, the pad 1160a is moved in the longitudinal direction, and the pad 1160b and the pull wire 1024 are moved along with it. Thus, by rotating the wheel 1134, the user may control the longitudinal position of the pull wire 1024 with respect to the outer shaft member 1022, and thereby control the extent to which the expandable member is radially deployed. The pads 1160a-f may be formed from C-Flex or Pebax and are preferably about 0.5-1.0″ long, 0.25-0.5″ wide, and 0.125-0.25″ thick.

The wheel 1134 imparts motion via a cam mechanism (not shown) to the pad 1160a which moves the pull wire 1024 incrementally. The wheel 1134 may advantageously move the pull wire 1024, for example, between 3 mm and 20 mm as indicated by a dial 1135 on the face of the wheel (see FIG. 63), thereby controlling the extent to which the expandable member is expanded by controlling the position of the pull wire. The dial 1135 acts as a gauge of the relative longitudinal position of the pull wire 1024 within the vessel, and thus as a gauge of the extent to which the expandable member has been expanded.

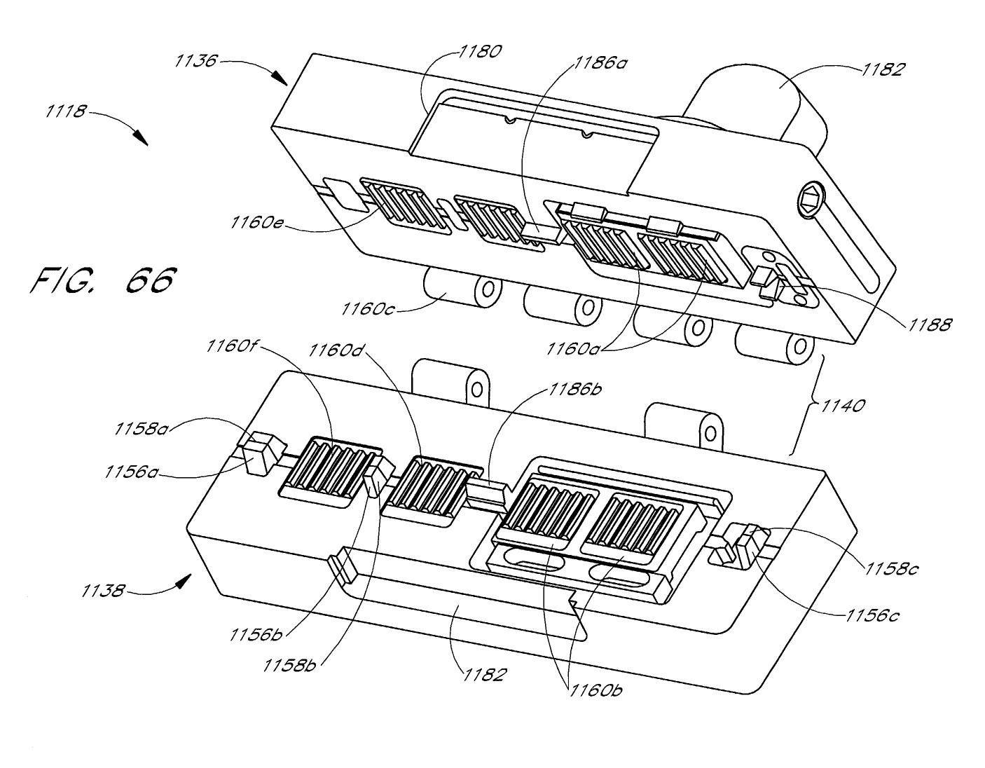

Another embodiment of the adapter 1118 is shown in FIG. 66. This embodiment has the same basic configuration as that shown in FIGS. 63-65, i.e., a clamshell with two halves 1136, 1138 rotatably connected by at least one hinge 1140. A resilient locking clip (not shown) may be mounted in a recess 1180 formed in the upper half 1136 and extend downward therefrom. Upon closure of the adapter 1118 an inwardly-extending tongue formed on the locking clip snaps into a groove 1182 formed in the lower half 1138. The locking clip holds the adapter 1118 firmly closed by virtue of an interference fit between the tongue and the groove 1182.

In place of the thumb wheel 1134 shown in FIGS. 63-65, this embodiment of the adapter 1118 incorporates a knob 1184 that is rotated by the user to move the pads 1160a, 1160b and advance/retract the pull wire 1024. Like the thumb wheel 1134, the knob 1184 may incorporate appropriate markings (not shown) to indicate the extent to which the filter has been expanded or retracted by the action of the adapter 1118.

Like the adapter shown in FIGS. 63-65, the adapter 1118 of FIG. 66 includes pads 1160c, 1160d, 1160e, 1160f that grip the outer shaft member and hold it stationary while the pull wire is advanced or retracted within it. Clips 1156a, 1156b, 1156c having respective slots 1158a, 1158b, 1158c receive the outer shaft member and/or pull wire and maintain it in a straight configuration for the filter deployment/retraction process. Upper and lower channel halves 1186a, 1186b coact to create, upon closure of the adapter 1118, a channel that receives and grips the outer shaft member and the pull wire, preferably immediately adjacent the pads 1160a, 1160b.

A pin member 1188 is positioned on the upper half 1136 so that the pin 1188 is depressed by the pull wire when the adapter 1118 is closed with the outer shaft member and pull wire positioned therein. The pin member is mechanically coupled to an interrupt mechanism (not shown) that prevents rotation of the knob 1182 unless the adapter 1118 is closed with the pull wire, etc. in position (and the pin member 1188 depressed by contact with the pull wire).

Additional details not necessary to repeat here are disclosed in assignee's copending application entitled OCCLUSION OF A VESSEL AND ADAPTER THEREFOR, application Ser. No. 09/505,911, filed Feb. 17, 2000, the entirety of which is hereby incorporated by reference.

Strut Design

With further reference to FIG. 56, the filter device includes a filter subassembly 1014 which is located along the shaft 1012 near the distal end, and proximal of the guide tip 1016. In one embodiment the filter subassembly may be integrally formed with the outer member 1022 of the shaft 1012. The filter subassembly 1014 comprises a number of struts 1028 and an occlusive member or membrane 1026. The struts support the membrane, and provide for at least two configurations of the device, a collapsed configuration and an expanded configuration. The expanded configuration is shown.

The “collapsed” configuration refers to the lowest profile configuration of the struts. In this context, “profile” refers to the distance away from the axis of the device that is spanned. Therefore, “low profile” refers to configurations in which the device is entirely within a small distance from the axis of the device. The “collapsed configuration” is the configuration in which the struts have the lowest possible profile, that is, where they lie as close as possible to the axis of the device. Having a low profile configuration simplifies insertion and removal of the device, and strut designs which tend to reduce the profile of the occlusion device are advantageous.

In the collapsed configuration, the embodiment shown in FIG. 56 would have the struts 1028 and the occlusive member 1026 positioned as close as possible to the longitudinal axis of the device, i.e. they would have the smallest possible cross-section. This configuration facilitates the deployment of the filter subassembly 14 by permitting easier delivery through the blood vessel 1018 on the distal end of a catheter shaft, as well as easier retrieval of the filter subassembly 1014 at the conclusion of the procedure. By minimizing the profile of the filter subassembly, this configuration is more easily passed through the vasculature leading to the filtration site from the insertion point.

When moved from the expanded configuration, shown in FIG. 56, into the collapsed configuration, the membrane 1026 may not lie in the same profile as it did prior to deployment into the expanded configuration. This is because the membrane is retracted strictly by the action of the struts, and excess folds of material may extend from between the struts in the collapsed configuration. This may cause the profile of the filter subassembly 1014 to be larger after retraction than it was prior to deployment. This enlarged profile can cause the membrane 1026 to rub against the vessel walls in an undesirable manner. One way to address this difficulty is to use a retrieval catheter as described in Applicant's copending application entitled STRUT DESIGN FOR AN OCCLUSION DEVICE, application Ser. No. 09/505,546, filed Feb. 17, 2000, the entirety of which is hereby incorporated by reference.

In the “expanded” configuration shown in FIG. 56, the struts 1028 and the occlusive member 1026 are positioned such that they span substantially the entire width of the blood vessel 1018 in which they are positioned. This is preferably the highest profile possible for the struts within the blood vessel. This configuration facilitates the use of the filter subassembly 1014 to trap embolic matter while permitting passage of blood through the filter subassembly. By providing a means to span substantially the entire width of the blood vessel 1018 to be filtered, the struts 1028 support the occlusive member 1026 in a configuration which forces the blood flow through the vessel to pass through the pores or openings in the filter subassembly 1014 while retaining emboli therein. This produces the desired filtering effect.

Actuation of the struts in order to adjust the device from the collapsed configuration to the expanded configuration (shown in FIG. 56) is achieved using either a tension or a torsion mechanism. In tension based actuation, the pull wire 1024 is displaced axially within the outer shaft member 1022 in a proximal direction. In one preferred embodiment, this displacement allows the struts to expand under a built-in bias into the expanded configuration. In the embodiment shown in FIG. 56, the displacement applies an outward biasing force to the struts. In torsion based actuation, the pull wire 1024 is rotated with respect to the outer shaft member 1022, resulting in a rotational displacement which applies an outward biasing force to the struts. In order to adjust from the expanded to the collapsed configuration, the actuation is reversed, by either pushing or rotating the pull wire in the direction opposite from that used in the deployment, reversing the force upon the struts, and returning the device to the original configuration. Additional details are disclosed in the above-referenced STRUT DESIGN FOR AN OCCLUSION DEVICE.

Membrane

As seen in FIG. 56, the occlusive member or membrane 1026 is preferably attached to each of the struts 1028 and extends completely around the longitudinal axis of the device. Preferably, the occlusive member 1026 is attached to the outer surface of the struts 1028; however, it may be attached along the inside of the struts 1028 as well. Moreover, it will be appreciated that the filter membrane may be provided inside some of the struts and outside of others. It will also be appreciated that struts may be provided on both sides of the membrane in a sandwiched configuration, or that two membranes may sandwich a set of struts.

At its distal end the occlusive member 1026 is preferably joined to the strut hypotube 1030, or, alternatively, to the guide tip 1016. As the occlusive member 1026 can be constructed in varying lengths, its proximal end may be located between the midpoint and the proximal end of the struts 1028. Where the occlusive member 1026 extends along the entire length of the struts 1028 it may also be attached at its proximal end to the strut hypotube 1030. Thus, when the struts 1028 are radially expanded, the occlusive member 1026 will likewise expand so as to take on a cross-sectional area corresponding approximately to that of the internal dimensions of the blood vessel 1018. It is contemplated that the occlusive member can be joined to the struts 1028 and strut hypotube 1030 by employing standard attachment methods, such as heat fusing, adhesive bonding, etc.

One preferred occlusive member 1026 is a nonelastomeric membrane with a number of pores which are approximately 20-100 microns in diameter. Suitable nonelastomeric materials include, but are not limited to: polyurethane, polyethylene, polyethylene terephthalate (PET), expanded polytetrafluoroethylene (PTFE), and polyether-based polyamides sold under the trade name PEBAX by Elf Atochem. This type of occlusive member may be extruded or dip molded, with the pores formed by the mold itself, or subsequently using an excimer laser or other drilling process.

One suitable elastomeric material is a block copolymer of styrene-ethylenebutylene-styrene (SEBS), available under the trade name C-FLEX, sold by Consolidated Polymer Technologies. The membrane may also be made from latex or silicone. The occlusive member may alternatively comprise a polymer mesh of polyurethane, nylon, polyester, or polyethylene, with pores approximately 30-50 microns in diameter. Yet another alternative is a braid of polyester or nitinol. To prevent formation of blood clots on the occlusive member, it may be coated with heparin or other known antithrombogenic agents such as hirudin or pirudin.

A variety of pore configurations are suitable for use with the occlusive member. First, where the membrane extends along the entire length of the struts, about 2-10 pores of about 20-200 microns diameter may be arranged longitudinally along the occlusive member. Another suitable configuration for this type of occlusive member consists of several pores of about 20-200 microns in diameter on the distal half of the member, and large triangular, round, or square cutouts on the proximal half. Alternatively, the entire surface of the occlusive member may have pores of about 20-200 micron size. This configuration is also contemplated for use where the occlusive member 1026 has an open proximal end. When using this type of occlusive member, a non-permeable cover or web may be placed over the juncture of the proximal ends of the struts to the distal shaft, to prevent formation of thrombi in the narrow passages formed at this point.

The membrane may be mounted on the device so as to create a loose or “baggy” portion of the membrane between proximal and distal points of attachment to the struts and to the strut hypotube/guide tip, respectively. In other words, the membrane may have a proximal point or region of attachment to the struts, a baggy portion distal of the proximal point of attachment in which the membrane is unattached to the device, and a distal point of attachment distal of the baggy portion. On such a membrane, the distal and proximal portions that are intended for attachment to the struts, guide tip and/or strut hypotube may preferably be substantially nonporous, to permit better adhesion.

The membrane or occlusive member may also comprise a strut-deployable balloon that incorporates perfusion tubes which permit fluid communication (but not flow of emboli) between the proximal and distal sides of the balloon. The perfusion tubes may comprise lengths of tubing which terminate (at their proximal and distal ends, respectively) at points of intersection with the proximal and distal faces of the balloon. Alternatively, perfusion may be facilitated through the lumen of the outer shaft member via openings formed therein proximal of the balloon, and via the (porous) guide tip distal of the balloon. A valve system may be employed to regulate the flow of fluid through the lumen.

The device may also employ dual occlusive members on a single set of struts, with a proximal filter with relatively large pores and a distal filter with smaller pores. With any of the mentioned types of occlusive member, it is contemplated that an aspiration catheter may be employed to remove thrombi from the filter(s) at various points in an angioplasty or other similar procedure.

Guide Tip

As shown in FIG. 56, located most distally upon the shaft 1012 is a guide tip 1016. The guide tip lies distal of the filter subassembly 1014 and provides a flexible leading extension which bends to follow the curvature of the blood vessels through which the device is advanced. By bending to follow the wall of the blood vessel, the guide tip 1016 leads the filter subassembly 1014 and other more proximal elements of the device in the direction of the tip so as to make the device move through the vessel without excessive impact against the walls of the blood vessels of the patient.

With further reference to FIG. 56, in one embodiment the guide tip 1016 is formed by creating a rounded solder joint tip 1035 to the pull wire 1024 of the shaft 1012, and wrapping it in a thinner wire to produce a coil which provides a spring force between the filter subassembly 1014 and the rounded tip 1035. The wire used for the coil 1016 is preferably made of a radiopaque material. Because the pull wire 1024 is constructed of a flexible material, such as nitinol, it will bend when the rounded tip 1035 is pushed against the curving wall of a blood vessel. However, as the deflection of the tip increases, the spring force of the coil of thinner wire will urge the filter subassembly 1014 and shaft 1012 into alignment with the guide tip 1016. In this way, the entire shaft is made to follow the path of the guide tip 16 as it advances through the blood vessels toward the treatment site.

Operation

The use of the described embodiments of the instant invention will generally be part of a process of therapy on a portion of the blood vessel of a patient. Usually, the therapy will involve treatment of some form of blockage of the blood vessel. However, those skilled in the art will recognize that the use of the described invention is appropriate in any situation where there is a possibility of embolic matter being dislodged from the vasculature of the patient, and therefore a desire to inhibit the dispersal of such embolic matter into the bloodstream of the patient.

As used herein, “method” refers to a preferred sequence used to accomplish a goal. Furthermore, the method which is described below is not limited to the exact sequence described. Other sequences of events or simultaneous performance of the described steps may be used when practicing the instant invention.

First, the device is manipulated so that the filter subassembly or subassemblies are in the collapsed position. This simplifies the insertion of the device into the blood stream of the patient. The device is then inserted through an insertion site into a blood vessel of the patient. Once inserted into the vasculature of the patient, the device is advanced distally until the distal portion of the device is located adjacent to the region of the blood vessel to be treated.

The device is positioned such that the filter subassembly lies generally downstream of the treatment site, or more generally, such that the filter subassembly lies between the treatment site and any site which is of particular susceptibility to embolic damage (e.g., the brain or coronary arteries). In this way, the filter is positioned so as to intercept any embolic matter dislodged at the treatment site, before such embolic material can reach any vulnerable area or be dispersed through the blood flow of the patient.

Once in position, the filter subassembly is actuated so that it assumes its expanded configuration, effectively occluding the blood vessel so that all blood flow must pass through at least one of the filter membranes or other occlusive members of the device.

The desired therapy is now performed upon the region of the blood vessel to be treated. This may involve placement or removal of support stents, balloon angioplasty, or any other vascular therapy that is conducted through the use of interventional techniques. In the course of such interventional treatment, additional catheters or other devices may be introduced to the treatment area by threading them over or along the shaft of the occlusive device. During the therapy, any embolic matter which is dislodged will flow into the filter and be caught by the membranes supported by the struts.

At any point during the therapy, the embolic matter may be aspirated from the filters through the use of separate aspiration catheters or through the lumen of the outer hypotubes forming the shaft of the occlusive device. Such aspiration may be repeated as often as necessary to maintain perfusive blood flow through the filter subassembly and treated region.

When the therapy is concluded, the filter subassembly is retracted into its collapsed configuration by reversing the actuation process. This will return the struts to a low profile which can then be withdrawn from the patient through the insertion site.

The above presents a description of the best mode contemplated for a medical wire introducer and protective sheath according to the present invention, and of the manner and process of making and using it, in such full, clear, concise, and exact terms as to enable any person skilled in the art to which it pertains to make and use this invention. The embodiments of the medical wire introducer and protective sheath described herein are, however, susceptible to modifications and alternate constructions which are fully equivalent. Consequently, it is not the intention to limit this medical wire introducer and protective sheath to the particular embodiments disclosed. On the contrary, the intention is to cover all modifications and alternate constructions coming within the spirit and scope of the invention as generally expressed by the following claims, which particularly point out and distinctly claim the subject matter of the present invention.

What is claimed is:

1. A protective sheath assembly for introducing a catheter into a blood vessel, comprising:
a protective sheath having a proximal end, a distal end, and an elongated body defining a lumen along a longitudinal axis of the protective sheath, said lumen being constructed so that the catheter can be moved through the lumen, wherein the proximal end of the protective sheath is constructed from a material different from the material used to construct the distal end of the protective sheath.

2. The protective sheath assembly of claim 1 wherein the material used to construct the proximal end of the protective sheath is relatively stiffer than the material used to construct the distal end of the protective sheath.

3. The protective sheath assembly of claim 1 wherein the material used to construct the proximal end of the protective sheath is relatively more flexible than the material used to construct the distal end of the protective sheath.

4. The protective sheath assembly of claim 1 wherein the proximal end of the protective sheath is constructed from a material selected from the following group of materials: PEBAX, PE, PEEK, FEP, PTFE, polyimide (Nylon), polycarbonate, and a silicone-based material.

5. The protective sheath assembly of claim 1 wherein the distal end of the protective sheath is constructed from a material selected from the following group of materials: PEBAX, PE, PEEK, FEP, PTFE, polyimide (Nylon), polycarbonate, and a silicone-based material.

6. The protective sheath assembly of claim 1 wherein the proximal end of the protective sheath is constructed from a silicone-based material and the distal end of the protective sheath assembly is constructed from PEBAX.

7. The protective sheath assembly of claim 1 further comprising a strain-relief tubing.

8. The protective sheath assembly of claim 7 wherein the strain-relief tubing is constructed from a material selected from the following group of materials: PEBAX, PE, PEEK, FEP, PTFE, polyimide (Nylon), polycarbonate, and a silicone-based material.

9. The protective sheath assembly of claim 1 further comprising a connecting member having a proximal end, a distal end, and a through-hole generally aligned with the longitudinal axis of the protective sheath.

10. The protective sheath assembly of claim 9 wherein the connecting member is constructed from a material selected from the following group of materials: PEBAX, PE, PEEK, FEP, PTFE, polyimide (Nylon), polycarbonate, and a silicone-based material.

11. The protective sheath assembly of claim 9 wherein the through-hole of the connecting member tapers in diameter from its proximal end to its distal end.

12. The protective sheath assembly of claim 9 wherein the through-hole of the connecting member tapers in diameter in a uniform manner from its proximal end to its distal end.

13. The protective sheath assembly of claim 1, wherein the lumen of the protective sheath is coated with a silicone coating.

14. The protective sheath assembly of claim 9, wherein the lumen of the protective sheath and the through-hole of the connecting member are coated with a silicone coating.

15. The protective sheath assembly of claim 7 further comprising a tubing surrounding a portion of the strain-relief tubing.

16. The protective sheath assembly of claim 15 wherein the tubing surrounding a portion of the strain-relief tubing is constructed from a material selected from the following group of materials: PEBAX, PE, PEEK, FEP, PTFE, polyimide (Nylon), polycarbonate, and a silicone-based material.

17. The protective sheath assembly of claim 9 further comprising a tubing surrounding a portion of the connecting member.

18. The protective sheath assembly of claim 17 wherein the tubing surrounding a portion of the strain-relief tubing is constructed from a material selected from the following group of materials: PEBAX, PE, PEEK, FEP, PTFE, polyimide (Nylon), polycarbonate, and a silicone-based material.

19. A protective sheath assembly for introducing a catheter into a blood vessel, comprising:
a protective sheath having a proximal end, a distal end, and an elongated body defining a lumen along a longitudinal axis of the protective sheath, said lumen being constructed so that the catheter can be moved through the lumen,
a strain-relief tubing, and
a connecting member having a proximal end, a distal end, and a through-hole generally aligned with the longitudinal axis of the protective sheath, wherein the through-hole of the connecting member tapers in diameter from its proximal end to its distal end.

20. The protective sheath assembly of claim 19 wherein the through-hole of the connecting member tapers in diameter in a uniform manner from its proximal end to its distal end.

21. The protective sheath assembly of claim 19, wherein the lumen of the protective sheath is coated with a silicone coating.

22. The protective sheath assembly of claim 19, wherein the lumen of the protective sheath and the through-hole of the connecting member are coated with a silicone coating.

23. The protective sheath assembly of claim 19 further comprising a tubing surrounding a portion of the strain-relief tubing.

24. The protective sheath assembly of claim 23 wherein the tubing surrounding a portion of the strain-relief tubing is constructed from a material selected from the following group of materials: PEBAX, PE, PEEK, FEP, PTFE, polyimide (Nylon), polycarbonate, and a silicone-based material.

25. The protective sheath assembly of claim 19 further comprising a tubing surrounding a portion of the connecting member.

26. The protective sheath assembly of claim 25 wherein the tubing surrounding a portion of the strain-relief tubing is constructed from a material selected from the following group of materials: PEBAX, PE, PEEK, FEP, PTFE, polyimide (Nylon), polycarbonate, and a silicone-based material.

27. A protective sheath assembly for introducing a catheter having a shaft and a distal device into a blood vessel, comprising:
an outer sheath having an elongated tubular body with a proximal portion and a distal portion, said elongated tubular body defining a lumen, said lumen being constructed so that said distal device of said catheter can be advanced through said lumen; and
an inner sheath having an elongated inner body defining a shaft lumen, said elongated inner body being insertable into said lumen in said proximal portion of said outer sheath, said shaft lumen being formed to accommodate said shaft of said catheter during introduction of said catheter into said blood vessel.

28. The protective sheath assembly of claim 27, wherein said inner sheath is formed with a longitudinal slot for providing access to said shaft lumen thereby allowing said shaft to be side-loaded into said shaft lumen.

29. The protective sheath assembly of claim 27, wherein said inner sheath provides a seal in said lumen of said outer sheath for minimizing blood back flow through said lumen in said outer sheath.

30. The protective sheath assembly of claim 27, further comprising a strain-relief tubing enclosing said proximal portion of said outer protective sheath.

31. The protective sheath assembly of claim 27, further comprising:
a mandrel lumen formed in said inner sheath; and
a mandrel contained in said mandrel lumen for providing structural support.

32. The protective sheath assembly of claim 27 wherein said inner sheath and said outer sheath are constructed from a material selected from the following group of materials: PEBAX, PE, PEEK, FEP, PTFE, polyimide (Nylon), polycarbonate, and a silicone-based material.

33. A protective sheath assembly for introducing a catheter having a shaft and a distal device into a blood vessel, comprising:
an outer sheath having an elongated tubular body defining a lumen along a longitudinal axis, said elongated tubular body defining a lumen, said lumen being constructed so that said distal device of said catheter can be advanced through said lumen, said elongated tubular body being formed with an outer slot such that said shaft may be side-loaded into said lumen; and
an inner sheath having an elongated inner body defining a shaft lumen along a longitudinal axis, said elongated inner body being insertable into said lumen in said outer sheath, said elongated inner body being formed with an inner slot such that said shaft may be side-loaded into said filter shaft,
whereby said inner sheath can be inserted into lumen of said outer sheath with said inner slot aligned with said outer slot such that said shaft may be side-loaded through both said outer and inner slots and into said filter shaft in said inner sheath.

34. The protective sheath assembly of claim 33, wherein said inner sheath is rotatable relative to said outer sheath to move said inner slot out of alignment with said outer slot thereby locking said shaft in said shaft lumen.

35. A protective sheath assembly for introducing a catheter into a blood vessel, comprising:
an outer sheath having an elongated tubular body with a proximal portion, a distal portion, and an entrance port, said elongated tubular body defining a lumen, said lumen being constructed so that said distal device of said catheter can be advanced through said lumen, said proximal portion of said elongated tubular body being formed with a proximal slot such that said shaft may be side-loaded into said lumen, said distal portion of said elongated tubular body being formed with a distal slot such that said shaft may be peeled out of said lumen, said entrance port being located between said proximal slot and said distal slot such that said distal end of said catheter may be inserted into said lumen; and
an inner sheath having an elongated inner body defining a shaft lumen, said elongated inner body being insertable into said lumen in said proximal portion of said outer sheath, said shaft lumen being formed to accommodate said shaft of said catheter during introduction of said catheter into said blood vessel, said elongated inner body being formed with a slot extending along said longitudinal axis for side-loading said shaft into said filter shaft.

36. The protective sheath assembly of claim 35, wherein said inner sheath is rotatable relative to said outer sheath to move said inner slot out of alignment with said outer slot thereby locking said shaft in said shaft lumen.

37. The protective sheath assembly of claim 35, further comprising:
a mandrel lumen formed in said inner sheath; and
a mandrel contained in said mandrel lumen for providing structural support.

38. A protective sheath assembly for introducing a catheter having a shaft and a distal end into a blood vessel, comprising an elongated tubular body having a proximal portion and a distal portion, said elongated tubular body defining a lumen constructed so that said catheter can be advanced through said lumen, said elongated tubular body being formed with an entrance port located between said proximal portion and said distal portion for inserting said distal end of said catheter into said lumen in said distal portion, said distal portion being formed with a slot for peeling said shaft out of said lumen through said slot.

39. A protective sheath assembly for introducing a catheter into a blood vessel, comprising an elongated tubular body defining a lumen, said elongated tubular being constructed so that said catheter can be moved through said lumen, said elongated tubular body being formed with a longitudinal slot for side-loading of said catheter into said lumen.

40. A protective sheath assembly for introducing a catheter having a shaft and a distal end into a blood vessel, comprising:
an elongated tubular body having a proximal end, a distal end, a proximal portion, a distal portion, and an entrance port, said distal portion defining a distal lumen, said proximal portion defining a shaft lumen, said entrance port being located between said proximal portion and said distal portion for inserting said distal end of said catheter into said distal lumen;
a distal slot extending longitudinally along said distal portion for peeling said shaft of said catheter out of said distal lumen; and
a proximal slot extending longitudinally along said proximal portion for side-loading said shaft into said shaft lumen and removing said shaft from said shaft lumen.

41. The protective sheath assembly of claim 40, further comprising:
a mandrel lumen formed in said proximal portion extending substantially parallel to said shaft lumen; and
a mandrel contained in said mandrel lumen for providing structural support.

42. The protective sheath assembly of claim 40, further comprising a hub coupled to said proximal end of said protective sheath, said hub defining a hub lumen, said hub being formed with a hub slot extending along said longitudinal axis to provide access to said hub lumen.

43. The protective sheath assembly of claim 42, wherein said hub is rotatable relative to said protective sheath whereby said hub slot may be moved out of alignment with said longitudinal slot so that said catheter cannot be removed from said hub lumen of said protective sheath assembly.

44. The protective sheath assembly of claim 40, further comprising a hub coupled to said proximal end of said protective sheath, said hub having a first portion and a second portion connected by a hinge whereby said first portion is moved out of engagement with said second portion such that said catheter may be placed into said hub lumen and said first portion is moved back into engagement with said second portion to lock said catheter in said hub lumen.

45. The protective sheath assembly of claim 40, further comprising a hub coupled to said proximal end of said protective sheath, said hub having two separable halves, wherein said separable halves are separated to place medical device into said hub lumen and said separable portions are pushed together to lock said medical device in said hub lumen.

46. The protective sheath assembly of claim 40, wherein said distal portion of said elongated tubular body is formed with pores for providing fluid communication across said distal portion of said elongated tubular body to flush air out of said elongated tubular body.

47. A protective sheath assembly for introducing an intravascular device into a blood vessel, comprising:
an elongated tubular body defining an interior lumen and having a proximal end portion, said lumen being constructed to allow said intravascular device to be advanced through said lumen;
a slot extending longitudinally along said proximal end portion of said tubular body, said tubular body having first and second opposing walls located on either side of said slot, said first and second opposing walls being in contact while said tubular body is in a relaxed condition such that said slot is closed; and
at least two projections extending radially from said proximal end portion which can be manipulated to deform said tubular body such that said first and second opposing walls become separated to open said slot for providing access to said lumen thereby allowing said intravascular device to be side-loaded into said lumen.

48. The protective sheath assembly of claim 47, further comprising a pair of guide members located on opposite sides of said slot and extending radially from said tubular body for facilitating loading of said intravascular device through said slot into said interior lumen.

49. The protective sheath assembly of claim 47, further comprising an annular member located within said interior lumen along said proximal end portion, said annular member providing a seal to prevent blood backflow through said tubular body, said annular member forming an orifice adapted to slidably receive said intravascular device.

50. The protective sheath assembly of claim 47 wherein said first and second opposing walls of said tubular body overlap along said slot when the sheath is in a relaxed condition.
